SEQUENCE LISTING (1 sequence)

SEQ ID NO: 1 - protein, 128 aa, unidentified
MAAAVEALFVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

(19) United States
(12) Patent Application Publication
(10) Pub. No.: US 2026/0224638 A1
(43) Pub. Date: Aug. 6, 2026

(54) Use Of Umbilical Vein Endothelial Cells For The Treatment Of Lung Injury

(71) Applicant: ANGIOCRINE BIOSCIENCE, INC., San Diego, CA (US)

(72) Inventors: Paul William Finnegan, Del Mar, CA (US); John K. Fraser, San Diego, CA (US)

(21) Appl. No.: 19/149,372

(22) PCT Filed: Jan. 18, 2024
(86) PCT No.: PCT/US24/11983

§ 371 Date: Jul. 18, 2025

Related U.S. Application Data
(60) Provisional application No. 63/480,489, filed on Jan. 18, 2023.

(51) Int. Cl. A61K 35/51; A61K 47/20; A61K 47/36; A61K 47/42; A61P 11/00

(52) U.S. Cl. CPC A61K 35/51; A61K 47/20; A61K 47/36; A61K 47/42; A61P 11/00

(57) ABSTRACT

The present invention relates to the use of umbilical vein endothelial cells (UVECs) for the treatment of lung injury and provides various methods and compositions relating thereto.

### CROSS-REFERENCE TO RELATED APPLICATIONS

This application claims the benefit of priority of U.S. Provisional Patent Application No. 63/480,489 filed on Jan. 18, 2023, the content of which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 17, 2024, is named Angio_033_WO1_SEQ LIST, and is 1867 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND OF THE INVENTION

A number of conditions lead to loss of pulmonary function through injury to or destruction of alveolar tissue. These include infectious diseases, such as COVID-19 disease, autoimmune diseases, environmental factors (e.g., cigarette smoke, dust, air pollution, toxins, and pro-fibrotic agents), and genetic factors (such as those predisposing individuals to autoimmune disease such as systemic sclerosis or to idiopathic pulmonary fibrosis). Surgical resection occurring, for example, as part of treatment for malignant disease of or metastasizing to the lung can also lead to loss of alveolar function. Loss of critical tissue at the alveolar: capillary barrier in these settings leads to reduced lung function with clinically-relevant sequelae including dyspnea, pulmonary edema, and hypoxemia. Restoration of pulmonary function is dependent upon generation of new alveolar: capillary tissue. This requires the activity of multiple cell types including alveolar epithelial cells, the stem and progenitor cells that give rise to alveolar epithelial cells, and several other cell types including mesenchymal cells, airway smooth muscle, and endothelial cells. When repair is effective alveolar structure and function are reestablished and pulmonary function is restored. However, for many individuals, the extent of injury, repeated or sustained injury or inflammation, underlying comorbidities, or other factors lead to delayed or impaired healing such that restoration of pulmonary function is unsatisfactory, and loss of lung function is at least partially irreversible. In such cases there is a clear need for interventions that can accelerate and/or enhance pulmonary regeneration and healing.

A number of studies have demonstrated that pulmonary endothelial cells are one of the key populations in pulmonary regeneration. For example, researchers have shown that infusion of endothelial cells derived from mouse lung accelerates repair of mouse lung following injury induced by instillation of the enzyme elastase (Hisata et al.) and enhances expansion of lung tissue following pneumonectomy (Ding et al., Cell. 2011 Oct. 28; 147(3):539-53). Further, Lee et al. have reported that co-culture of lung stem cells with endothelial cells supported stem cell self-renewal (proliferation without loss of colony-forming capacity) and multilineage differentiation (Lee et al., 2014 Cell 156 (3) 440-455). They further reported that, while in vivo recovery of alveolar epithelial type 2 cells following Bleomycin-induced lung injury was impaired in thrombospondin-negative (Tsp1^{-/-}) mice, this capacity was recovered by administration of medium conditioned by endothelial cells obtained from wild-type (Tsp1^{+/+}) mice. Further, medium conditioned by endothelial cells from wild-type mice also reduced fibrosis in Tsp1^{-/-} mice restoring their BASC numbers to values comparable to those in wild-type mice. Importantly, in each of these cases, the capacity of the endothelial cells to mediate improved repair and regeneration was dependent upon the tissue from which the endothelial cells were obtained. Specifically, improved lung repair and regeneration was only evident when the endothelial cells were derived from the lung: repair of the lung was not mediated by infusion of endothelial cells from other tissues. Thus, Ding et al. reported that while infusion of pulmonary endothelial cells promoted lung regeneration following pneumonectomy, infusion of endothelial cells from the liver did not.

Similarly, Hisata et al reported that lung repair following elastase injury was promoted by infusion of pulmonary endothelial cells but not by endothelial cells obtained from adipose tissue. “Elastase-induced [endothelial cell] loss (VE-cadherin+ cells) was mitigated by injection of lung ECs^{E4ORF1}, but not lung fibroblasts or, importantly, nonlung [endothelial cells], such as adipose-derived [endothelial cells].” Also, “Likewise, only delivery of lung ECs^{E4ORF1} perturbed the increase in inspiratory capacity and reduction in lung elastance observed in mice treated with elastase only. These findings support previous reports that underscored the importance of organ-specific ECs in tissue repair and function.” (Hisata et al. page 4, column 2, first paragraph, see also Hisata et al FIGS. 4E and S3A).

Similarly, Lee et al reported that, bronchioalveolar colonies arising from co-culture of bronchoalveolar stem cells (BASC) with lung endothelial cells (“LuMECs”) when subcutaneously injected with liver endothelial cells (“LiEMCs) yielded primarily bronchiolar structures whereas when the same cells were co-injected with LuMECs, they generated three different types of lung epithelial structures (see Lee et al., page 4, first paragraph and FIGS. 3E and 3F). As with Ding et al. and Hisata et al., this tissue-of-origin specificity was evident in vivo. Specifically, the authors noted that lung progenitor (AT2) cell regeneration is impaired following bleomycin-induced lung injury model in thrombospondin-null mice. However, whereas administration of medium conditioned by wild-type lung ECs (LuMECs) led to AT2 cell regeneration that was comparable to wild-type mice, administration of medium conditioned by wild-type liver ECs (LiMECs) did not (Lee et al., page 7, paragraph 3; Figure S6).

This requirement for tissue-specificity substantially limits the potential application of endothelial cell-based therapy for enhancement of pulmonary regeneration in that treatment of human pulmonary disease or injury would require isolation and clinical expansion of endothelial cells derived from human lung. This would only be feasible and ethical using either allogeneic endothelial cells from cadaveric donor lungs (as in lung transplantation) or autologous endothelial cells biopsied from a patient's own lung tissue; a procedure with substantial practical limitations including the amount of tissue that can be obtained without inducing unacceptable morbidity and the fact that endothelial cells sourced from patients with lung disease or injury frequently exhibit dysfunction (Green and Turner 2017: Respir Res 18, 20 (2017). https://doi.org/10.1186/s12931-017-0505-1). Therefore, in order for endothelial cell-mediated lung therapy to be practical, a non-lung endothelial cell source will be required.

### SUMMARY OF THE INVENTION

The present invention is based, in part, on certain surprising discoveries that are described in the Examples section of this patent disclosure. In particular, it has now been discovered that endothelial cell-mediated enhancement of pulmonary repair can be achieved using endothelial cells other than those obtained from the lung. In particular, it has been discovered that the requirement for tissue-specific endothelial cells reported by Ding et al., Hisata et al., and Lee et al., can be overcome by use of endothelial cells derived from the umbilical vein. We provide herein data demonstrating that administration of compositions comprising umbilical vein endothelial cells (UVECs) to living subjects with lung injury results in structural and functional lung regeneration/repair—as demonstrated by improvements in a multitude of symptoms and indicators of lung injury. Building on these surprising discoveries, the present invention provides various new and useful methods and compositions, as summarized below and as described in additional detail in other sections of this patent disclosure.

Accordingly, in some embodiments the present invention provides methods for the treatment of lung injury in mammalian subjects in need thereof, the methods comprising administering to the subjects an effective amount of a composition comprising umbilical vein endothelial cells (UVECs). Similarly, in some embodiments the present invention provides methods for improving lung regeneration/repair in subjects in need thereof, the methods comprising administering the subjects an effective amount of a composition comprising umbilical vein endothelial cells (UVECs). In some such embodiments the umbilical vein endothelial cells (UVECs) are human umbilical vein endothelial cells (HUVECs). In some such embodiments the UVECs or HUVECs are E4ORF1+. In some embodiments the subjects are human. In some embodiments the UVECs (e.g., E4ORF1+HUVECs) are allogeneic with respect to the subject to whom they are administered.

In some embodiments the subjects have chronic obstructive pulmonary disease (COPD) and the methods result in treatment of the COPD in the subjects.

In some embodiments the subjects have emphysematous chronic obstructive pulmonary disease (COPD) and the methods result in treatment of the emphysematous COPD in the subjects.

In some embodiments the subjects have mild (GOLD 1) chronic obstructive pulmonary disease (COPD) and the methods result in treatment of the mild COPD in the subjects.

In some embodiments the subjects have moderate (GOLD 2) chronic obstructive pulmonary disease (COPD) and the methods result in treatment of the moderate COPD in the subjects.

In some embodiments the subjects have severe (GOLD 3) chronic obstructive pulmonary disease (COPD) and the methods result in treatment of the severe COPD in the subjects.

In some embodiments the subjects have very severe (GOLD 4) chronic obstructive pulmonary disease (COPD) and the methods result in treatment of the very severe COPD in the subjects.

In some embodiments the subjects have mild-to-moderate (GOLD 1-2) emphysematous chronic obstructive pulmonary disease (COPD) and the methods result in treatment of the mild-to-moderate COPD in the subjects.

In some embodiments the subjects have moderate-to-severe (GOLD 2-3) emphysematous chronic obstructive pulmonary disease (COPD) and the methods result in treatment of the moderate-to-severe COPD in the subjects.

In some embodiments the subjects have acute exacerbation of chronic obstructive pulmonary disease (AECOPD) and the methods result in treatment of the AECOPD in the subjects.

In some embodiments the subjects have lung injury associated with viral infection, and the methods result in treatment of the viral infection-associated lung injury in the subjects.

In some embodiments the subjects have lung injury associated with SARS-CoV2 infection and COVID19 disease, and the methods result in treatment of the SARS-CoV2 infection-associated lung injury in the subjects.

In some embodiments the subjects have lung injury associated with influenza virus infection, and the methods result in treatment of the influenza infection-associated lung injury in the subjects.

In some embodiments the subjects have lung injury associated with resipiratory syncytial viral (RSV) infection, and the methods result in treatment of the RSV infection-associated lung injury in the subjects.

In some embodiments the subjects have lung injury associated with acute respiratory distress syndrome (ARDS), for example resulting from SARS-CoV2, influenza or RSV infection, and the methods result in treatment of the ARDS in the subjects.

In some embodiments the subjects have pulmonary fibrosis, and the methods result in treatment of the pulmonary fibrosis in the subjects.

In some embodiments the subjects have Idiopathic Pulmonary Fibrosis (IPF), and the methods result in treatment of the IPF in the subjects.

In some embodiments the subjects have lung injury associated with physical trauma to the lung, and the methods result in treatment of the lung injury associated with the physical trauma in the subjects.

In some embodiments the subjects have lung injury associated with blunt trauma to the lung, penetrating trauma to the lung, or burn trauma affecting the lung, and the methods result in treatment of the lung injury associated with such trauma in the subjects.

In some embodiments the subjects have lung injury associated with surgical lung resection, and the methods result in treatment of the lung injury associated with the surgical lung resection in the subjects.

In some embodiments the composition comprising the UVECs is administered to the subjects intravenously (IV).

In some embodiments the composition comprising the UVECs is administered to the subjects in a single intravenous (IV) infusion.

In some embodiments the composition comprising the UVECs is administered to the subjects in multiple intravenous (IV) infusions.

In some embodiments the composition comprising the UVECs is administered to the subjects at a dose of from about 1×10⁶ cells per kg bodyweight to about 20×10⁶ cells per kg bodyweight, per infusion.

In some embodiments the composition comprising the UVECs is administered to the subjects at a dose of about 1×10⁶ cells per kg bodyweight, per infusion.

In some embodiments the composition comprising the UVECs is administered to the subjects at a dose of about 2.5×10⁶ cells per kg bodyweight, per infusion.

In some embodiments the composition comprising the UVECs is administered to the subjects at a dose of about 5×10⁶ cells per kg bodyweight, per infusion.

In some embodiments the composition comprising the UVECs is administered to the subjects at a dose of about 10×10⁶ cells per kg bodyweight, per infusion.

In some embodiments the composition comprising the UVECs is administered to the subjects at a dose of about 20×10⁶ cells per kg bodyweight, per infusion.

In some embodiments the present invention provides methods for the expansion of pulmonary tissue in vitro, for example following the resection of lung tissue from a subject, such methods comprising culturing the pulmonary tissue in vitro with a population of umbilical vein endothelial cells (UVECs). In some such embodiments the umbilical vein endothelial cells (UVECs) are human umbilical vein endothelial cells (HUVECs). In some such embodiments the UVECs or HUVECs are E4ORF1+.

In some embodiments the present invention also provides compositions useful in performing the methods of the present invention. In some embodiments the compositions comprise UVECs (such as HUVECs or E4ORF1+ HUVECs) and a physiological saline. In some such embodiments the compositions comprise one or more additional components suitable for administration to living subjects selected from the group consisting of buffers, salts, polysaccharides, proteins, and preservatives. In some such embodiments the compositions comprise one or more of human serum albumin (HSA), Dextran40, and up to about 0.25% DMSO. In some such embodiments the compositions comprise UVECs at a concentration of about 5 million cells per ml.

These and other embodiments of the invention are described further in other sections of this patent disclosure, and this Summary of the Invention is intended to be read in conjunction with all other sections of this patent disclosure. In addition, as will be apparent to those of skill in the art, certain modifications and combinations of the various embodiments described herein fall within the scope of the present invention.

### BRIEF DESCRIPTION OF THE DRAWINGS

FIG. 1. Graph showing effects of E-CEL UVEC treatment on inspiratory capacity in an acute COPD Model, as further described in the Examples section of this disclosure.

FIG. 2. Graph showing effects of E-CEL UVEC treatment on elastance in a chronic COPD Model, as further described in the Examples section of this disclosure.

FIG. 3. Graph showing lung endothelial cell content on day 10 following IV administration of E-CEL UVECs on day 7, as further described in the Examples section of this disclosure.

FIG. 4. Graph showing lung endothelial cell content on day 5 following IV administration of E-CEL UVECs on day 3 and day 4, as further described in the Examples section of this disclosure.

FIG. 5. Graph showing lung endothelial cell numbers on day 5 following IV administration of E-CEL UVECs on day 3 and day 4, as further described in the Examples section of this disclosure.

FIG. 6. Graph showing lung bronchoalveolar stem cell (BASC) content on day 5 following IV administration of E-CEL UVECs on day 2 and day 3, as further described in the Examples section of this disclosure.

FIG. 7. Graphs showing that E-CEL UVEC treatment is associated with reduced inflammation, fibrosis, and hemorrhage in a lung injury model, as further described in the Examples section of this disclosure.

### DETAILED DESCRIPTION

The “Summary of the Invention,” “Examples,” and “Claims” sections of this patent disclosure describe many of the main embodiments of the present invention. This “Detailed Description” section provides certain additional description relating to the compositions and methods of the present invention and is intended to be read in conjunction with all other sections of this patent disclosure. Furthermore, and as will be apparent to those in the art, the different embodiments described throughout this patent disclosure can be combined in different ways, regardless of any subheadings. Such combinations of the specific embodiments described herein fall within the scope of the present invention.

### Definitions

Certain definitions and abbreviations are provided below. Other terms or phrases may be defined elsewhere in this patent disclosure or may have meanings that are clear from the context in which they are used. Unless defined otherwise herein, or unless some other meaning is clear from their use in context herein, all technical and scientific terms and abbreviations used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10.

Wherever embodiments are described with the language “comprising,” otherwise analogous embodiments described in terms of “consisting of” and/or “consisting essentially of”′ are included.

As used herein, the terms “about” and “approximately,” when referring to a stated numeric value, indicate a value within plus or minus 10% of the stated numeric value In all embodiments where a numerical value is described using the terms “about” or “approximately” the analogous embodiment where the precise numerical value is used is also contemplated. For example, for an embodiment that involves a time of about 24 hours, the analogous embodiment involving a time of 24 hours (i.e., without the “about” qualification) is also contemplated As used herein, cells are “positive” or “+” for a started marker when that marker is expressed to a detectable degree in the cells. For example, endothelial cells are positive for, e.g., CD31 (i.e., are CD31+) if CD31 is expressed to a detectable degree by the endothelial cells, or are positive for E4ORF1 (i.e., are E4ORF1+) if E4ORF1 is expressed to a detectable degree by the endothelial cells. In some situations, expression of a stated marker may be assessed in comparison to, e.g., an experimental negative control.

As used herein, the abbreviation “E4ORF” refers to an open reading frame (ORF) present within the adenoviral early 4 (E4) region of an adenovirus genome, or a polypeptide/protein encoded by that ORF (whether the gene or the protein is referred to will be clear from the context of use). As used herein, the abbreviation “E4ORF1” refers to open reading frame (ORF) 1 of the early 4 (E4) region of an adenovirus genome, or a polypeptide/protein encoded by that ORF (whether the gene or the protein is referred to will be clear from the context of use).

As used herein, the abbreviation “E4ORF1+” when used with reference to a stated cell type refers to a cell that expresses E4ORF1 to a detectable degree. E4ORF1+ cells contain a recombinant E4ORF1 nucleic acid molecule and express the E4ORF1 protein.

As used herein, the abbreviation “EC(s)” refers to an endothelial cell(s).

As used herein, the abbreviation “UVEC” refers to umbilical vein endothelial cells.

As used herein, the abbreviation “HUVEC” refers to human umbilical vein endothelial cells.

As used herein, the terms “E-CELs” and “E-CEL UVECs” refer to E4ORF1+HUVECs or compositions comprising E4ORF1+ HUVECs. Whether the cells per se or a composition comprising the cells is referred to will be clear from the context in which the terms are used. For example, reference to E-CELs in the context of administration to a subject necessarily denotes administration of a composition comprising the E-CELs.

As used herein, the term “allogeneic” means deriving from, originating in, or being members of the same species, where the members are genetically related or genetically unrelated but genetically similar. The compositions and methods of the present invention preferably comprise or are performed using allogeneic UVEC cells. In such embodiments the allogeneic UVECs are obtained from a donor of the same species as the subject to whom the cells or composition will be administered (i.e., the recipient). In some embodiments the allogeneic cells are obtained from a donor having the a full or partial MHC/HLA match to the subject to whom the cells will be administered (i.e., the recipient)—i.e., the donor of the cells and the recipient of the cells are fully or partially MHC-matched or HLA-matched. In some embodiments, the UVECs are: (a) obtained from a donor, (b) maintained and/or cultured and/or expanded ex vivo, and (c) subsequently administered into a subject of the same species as the donor.

As used herein, the term “autologous” means deriving from or originating in the same subject. For example, in embodiments involving administration of autologous UVECs to a subject, the autologous cells are obtained from the subject to whom they will be administered (i.e., the donor and recipient of the UVECs are the same individual). In some embodiments, UVECs are: (a) obtained from a subject, (b) maintained and/or cultured and/or expanded ex vivo, and (c) subsequently administered to the same subject.

As used herein, the terms “treat,” “treating,” and “treatment” refer to achieving, to a detectable degree, an improvement in, or prevention of worsening of, one or more symptoms or sequelae of the lung injury being treated. For example, such terms include achieving, to a detectable degree (and optionally to a statistically significant degree and/or to a clinically significant degree), one or more of the following “treatment outcomes:” (a) improvement (i.e., increase) in, or prevention of worsening of, of lung function (e.g., as assessed using spirometry), (b) improvement (i.e., increase) in, or prevention of worsening of, Forced Expiratory Volume (FEV), (c) improvement (i.e., increase) in, or prevention of worsening of, Forced Vital Capacity (FVC), (d) improvement (i.e., increase) in, or prevention of worsening of, inspiratory volume, (e) improvement (i.e., increase) in, or prevention of worsening of, functional residual capacity (FRC), (f) improvement (i.e., increase) in, or prevention of worsening of, diffusion capacity for carbon monoxide (DLCO), (g) improvement (i.e., increase) in, or prevention of worsening of, tidal volume, (h) improvement (i.e., increase) in, or prevention of worsening of, total lung capacity, (i) improvement (i.e., decrease) in, or prevention of worsening of dyspnea, (k) improvement (i.e., increase) in, or prevention of worsening of, endothelial function (e.g., as assessed using vascular tonometry testing or using serum/plasma biomarkers), (1) improvement (i.e., increase) in, or prevention of worsening of, pulmonary microvascular blood flow and volume, (m) improvement (i.e., decrease) in, or prevention of worsening of, emphysema (e.g., as assessed using a high-resolution computed tomography (HRCT) scan), (n) improvement (i.e., increase) in, or prevention of worsening of, aerobic capacity (e.g., as assessed using a 6-minute walk test), (o) improvement (i.e., increase) in, or prevention of worsening of, aerobic endurance (e.g., as assessed using a 6-minute walk test), (p) improvement in, or prevention of worsening of, pulmonary physiology (e.g., as assessed using blood tests), (q) improvement (i.e., decrease) in, or prevention of worsening of, pulmonary edema, (r) improvement in, or prevention of worsening of, lung elastance, (s) improvement (i.e., decrease) in, or prevention of worsening of, lung inflammation, (t) improvement (i.e., decrease) in, or prevention of worsening of, lung fibrosis, (u) improvement (i.e., decrease) in, or prevention of worsening of, lung hemorrhage, (v), regeneration of, or reduction of loss of, lung tissue (e.g., as assessed using magnetic resonance imaging (MRI)), (w) regeneration of, or reduction of loss of, alveolar tissue (e.g., as assessed MRI), (y) expansion of bronchoalveolar stem cells, and/the like. Any suitable methods or clinical tests for assessing these treatment outcomes may be used and suitable methods for assessing such treatment outcomes will be well known to physicians practicing in the art of the present invention. In some embodiments, such treatment outcomes may be assessed by comparing a symptom or sequela in a single subject before and after treatment, or by comparing a symptom or sequela in a subject treated with a method as disclosed herein to that in a different individual who is not treated or is not treated or who is treated with a placebo, or by comparing a symptom or sequela in subject treated with a method as disclosed herein to a reference value for that symptom or sequela (e.g., a reference value for that symptom or sequela typically exhibited in a healthy person of the same sex, age and weight). For each of the embodiments described herein that refer to a method of treatment, an analogous method directed to achieving any one or more of the treatment outcomes listed above, or that results in any one or more of the treatment outcomes listed above, is also provided. For example, in addition to providing methods useful for the treatment of lung injury in subjects in need thereof comprising administering to the subjects an effective amount of a composition comprising umbilical vein endothelial cells (UVECs), the present invention also comprises an analogous method for the treatment of lung injury in subjects in need thereof comprising administering to the subjects an effective amount of a composition comprising umbilical vein endothelial cells (UVECs) wherein the treatment results in an improvement in Forced Expiratory Volume (FEV) in the subject, and also comprises an analogous method for improving Forced Expiratory Volume (FEV) in a subject with lung injury comprising administering to the subject an effective amount of a composition comprising umbilical vein endothelial cells (UVECs).

As used herein the term “effective amount” refers to an amount of a stated cell type (e.g., E4ORF1+ HUVECs), or a composition comprising that stated cell type, that is sufficient to achieve the stated outcome (e.g., treatment of lung injury) to a detectable degree, or, in some cases to a specifically stated degree. In some embodiments the effective amount is specified (e.g., in terms of a number of E4ORF1+ HUVECs per kg bodyweight). In other embodiments an appropriate “effective amount” may be determined empirically, for example using standard techniques known in the art, such as dose escalation studies, and may be determined taking into account such factors as the planned route of administration, desired frequency of administration, etc. Furthermore, an “effective amount” may be determined using studies such as those described in the Examples section of this patent disclosure.

The term “engineered” when used in relation to UVECs refers to UVECs cells that have been engineered by man to result in the recited phenotype (e.g., E4ORF1 expression), or to express a recited protein. The term “engineered cells” is not intended to encompass naturally occurring cells, but is, instead, intended to encompass, for example, cells that comprise a recombinant nucleic acid molecule, or cells that have otherwise been altered artificially, for example so that they express a protein that they would not otherwise express, or so that they express a protein at substantially higher levels than that observed in non-engineered UVECs.

As used herein the abbreviation “HLA” refers to human leukocyte antigen.

As used herein the abbreviation “IV” refers to intravenous.

As used herein term “subject” refers to a mammalian individual. In some embodiments the subject is a human. When the subjects are human the terms “subject” and “patient” may be used interchangeably.

As used herein, the term “lung injury” refers to a condition associated with either loss of lung tissue and/or reduction of lung function. Thus, the term “lung injury” as used herein encompasses “lung diseases” that are associated with loss of lung tissue and/or reduction of lung function, aging-related loss of lung tissue and/or reduction of lung function, and loss of lung tissue and/or reduced lung function that occurs as the result of physical “trauma” or “traumatic injury.”

The term “lung diseases” as used herein, includes, but is not limited to, infectious diseases (e.g., diseases caused by viral, bacterial, or fungal pathogens), autoimmune diseases, genetic diseases, diseases caused by environmental factors (such as air pollution, exposure to inhaled toxic substances, or smoking), cancer affecting the lungs, and the like.

The terms “trauma” and “traumatic injury” as used herein, include, but are not limited to, injury caused by blunt trauma, penetrating trauma, surgical trauma, and burns. Blunt trauma includes injuries caused by falls, road traffic crashes, crush injuries, and physical assaults. Penetrating trauma includes injuries caused by gunshots, knife wounds, and the like. Surgical trauma includes trauma resulting from surgical resection of tissue from the lung.

The different types of lung injury are not mutually exclusive. For example, in some instances a lung injury resulting from physical trauma lung may be considered a lung disease, or may lead to a lung disease, and vice versa.

As used herein, the term “autoimmune disease” refers to a disease in which the innate and/or adaptive immune system reacts abnormally towards tissues or cells within a subject.

### Lung Injury

The present invention provides compositions and methods useful for the treatment of lung injury. A non-limiting description of some of the types of lung injury (including, but not limited to, lung diseases) that can be treated is provided below.

Lung injuries include conditions affecting the organs and tissues that comprise the respiratory system including, but not limited to, the trachea, bronchi, bronchioles, alveoli, pleurae, pleural cavity, and the nerves and muscles of respiration.

Examples of lung injuries that can be treated using the methods and compositions of the present invention include, but are not limited to, lung diseases associated with infection with: a corona virus (such as severe acute respiratory syndrome associated coronavirus (SARS-Cov), severe acute respiratory syndrome associated coronavirus 2 (SARS-Cov2, the virus that causes COVID-19 disease), or Middle East Respiratory Syndrome coronavirus (MERS-COV)), acute respiratory distress syndrome, bacterial pneumonia, pulmonary embolism, pulmonary fibrosis, injury from toxic agents (such as chemotherapy agents or inhaled gases, thermal injury (inhalation injury), tuberculosis, acute asthma, and lung cancer. In many of these injury states there is damage to (and/or loss of) the critical tissues that form the key interface across which gases are transferred from inspired air into the bloodstream and vice versa. In such cases, restoration of pulmonary function requires a regenerative response in which new functional alveolar tissues are formed or injured tissue is remodeled (for example, by remodeling of fibrosis). However, in many situations the loss of pulmonary function is, at best, only partially reversible.

This lack of reversibility may be attributable to the combination of ongoing inflammatory injury and to impairment of the mechanisms responsible for repair and regeneration. These mechanisms include several different cell types including alveolar type II cells (AT2 cells; which can self-renew in culture and differentiate into both type II and type I epithelial cells) and bronchoalveolar stem cells (BASCs). Pulmonary endothelial cells also contribute to repair by forming the tissue-specific niche required for optimal function of lung regenerative mechanisms.

For example, Staudt et al., (Am J Respir Crit Care Med. 2014 Oct. 15; 190(8): 955-958) have reported that airway basal stem/progenitor cells have diminished capacity to regenerate airway epithelium in COPD. Chronologic aging is also associated with impaired lung homeostasis and repair. For example, in a review on aging of the lung and its impact on COPD, the authors that from age ~20 in females and ~25 in males, lung function progressively declines with increasing age (Brandsma et al., 2017, Eur Respir Rev. 2017 Dec. 6; 26(146):170073). These observations have led to studies showing that delivery of exogenous cells can supplement and enhance lung repair and regeneration (reviewed in Akram et al., Int J Mol Sci. 2016 Jan. 19; 17(1):128). While most studies have addressed cells capable of directly generating new pulmonary epithelial cells (pulmonary stem and progenitor cells, embryonic stem cells, induced pluripotent stem cells) others have examined cells that provide the regenerative niche within which pulmonary stem and progenitor cells exert their capacity to produce new epithelial cells. This includes examination of the role of pulmonary endothelial cells in lung regeneration. The role of tissue-specific vascular endothelial cells in providing a critical niche for repair and regeneration is common throughout the body. In a seminal review on this subject a leading group of authors stated: “Tissue-specific endothelium establish specialized vascular niches that deploy specific sets of growth factors, known as angiocrine factors, which actively participate in inducing, specifying, patterning, and guiding organ regeneration and maintaining homeostasis and metabolism.”

As discussed in further detail below, without being limited by a particular theory, the endothelial cells disclosed herein are not subject to this tissue-specificity, such that they promote recovery of lung function following injury or disease despite not being derived from lung but, rather, are derived from the umbilical cord. These cells promote restoration of the injured endothelium and supplement a microenvironment in which cells responsible for pulmonary tissue repair are more effective, and/or by directly or indirectly stimulating repair mechanisms. As such, the methods and compositions disclosed herein can advantageously enhance lung repair such that the patient so treated experiences accelerated restoration of lung function, improved lung function, reduced incidence and/or severity of subsequent pulmonary events that can exacerbate injury, and reduced incidence of subsequent events outside of the respiratory system.

In one embodiment the lung injury that is treated by the methods and compositions of the present invention is that caused by chronic obstructive pulmonary disease (COPD).

In one embodiment the lung injury that is treated by the methods and compositions of the present invention is that caused by acute exacerbation of chronic obstructive pulmonary disease (AECOPD).

In one embodiment the lung injury that is treated by the methods and compositions of the present invention is that caused by COVID-19. In one such embodiment, the injury treated arises from the acute effects of infection with the SARS-CoV2 virus. In another such embodiment, the injury treated arises from the chronic or delayed effects of SARS CoV2 infection (sometimes referred to as Long COVID Syndrome).

In one embodiment, the lung injury that is treated by the methods and compositions of the present invention arises from deposition of fibrous tissue within the respiratory system. In one such embodiment, the fibrous tissue arises from an autoimmune disease, such as systemic sclerosis. In another such embodiment, the fibrous tissue is associated with the disease Idiopathic Pulmonary Fibrosis (IPF). In another such embodiment, the fibrosis arises from exposure to a toxic agent, such as a chemotherapy drug (e.g.: bleomycin).

### Chronic Obstructive Pulmonary Disease (COPD)

In some embodiments, the lung injury treated by the methods and compositions of the present invention is associated with Chronic Obstructive Pulmonary Disease (COPD). COPD is a progressive chronic inflammatory lung disease characterized by labored breathing due to obstructed airflow, chronic bronchitis, destruction of small airways, and emphysematous destruction of alveolar tissue. It is frequently associated with significant extrapulmonary effects and comorbidities that may contribute to disease severity. Alveolar destruction in COPD results in marked airspace enlargement with reduction of alveolar: capillary exchange surface area. However, injury extends beyond the immediate alveolar: capillary interface with evidence of vascular dysfunction and denudation of endothelium present in larger pulmonary vessels of patients with COPD. (Yang et al., 2008 Curr. Drug Metab. 2008 September; 9(7):661-7; Peinado et al., Am J Respir Cell Mol Biol. 2006 March; 34(3):257-63. It is also evident that the pulmonary component is not fully reversible but rather exhibits slow progressive worsening punctuated by episodes of acute rapid deterioration. The absence of reversibility arises from the combination of ongoing inflammation as well as injury to (and exhaustion of) mechanisms responsible for pulmonary repair and regeneration. COPD may be classified clinically on the widely used GOLD grading scale (see, for example, Vestbo et al., “Global strategy for the diagnosis, management, and prevention of chronic obstructive pulmonary disease: GOLD executive summary;” Am. J. Respir. Crit. Care Med; 2013 Feb. 15; 187(4):347-65). According to the GOLD 1-4 system, COPD is graded based on the severity of airflow limitation, where GOLD 1 (mild) COPD is characterized by a forced expiratory volume in 1 second (FEV₁) of greater than or equal to 80% of the predicted value, GOLD 2 (moderate) COPD is characterized by an FEV₁ in the range from 50% to less than 80% of the predicted value, GOLD 3 (severe) COPD is characterized by an FEV₁ in the range from 30% to less than 50% of the predicted value, and GOLD 4 (very severe) COPD is characterized by an FEV₁ below 30% of the predicted value. The methods and compositions of the present invention may be used to treat COPD, emphysematous COPD, mild (GOLD 1) COPD, moderate (GOLD 2) COPD, severe (GOLD 3) COPD, or very severe (GOLD 4) COPD.

### Acute Exacerbation of COPD (AECOPD)

In some embodiments, the lung injury treated by the methods and compositions of the present invention is that associated with Acute Exacerbation of Chronic Obstructive Pulmonary Disease (AECOPD). As noted above, COPD is associated with a slow progressive decline in lung function punctuated by episodes of acute rapid worsening (exacerbation) often associated with respiratory infection or other insult. These acute exacerbations are defined as acute episodes of symptom worsening (over and above the day-to-day variations) that require medical intervention (change in regular medications). Susceptibility to exacerbations varies from patient-to-patient associated with differences in the underlying pathology, infection, immune status, medications, and comorbidities. Environmental agents such as air pollutants and second-hand smoke may also trigger acute exacerbations.

### COVID-19

In some embodiments, the lung injury treated by the methods and compositions of the present invention is that associated with COVID19 disease, resulting from infection with the SARS CoV2 corona virus, which is associated with severe acute and chronic lung injury. This is a consequence of the ability of the virus responsible for this disease to infect airway epithelial cells and vascular endothelial cells through the ACE2 receptor molecule. Infected patients develop rapidly evolving severe acute respiratory distress syndrome (ARDS) characterized by diffuse damage to the alveolar-capillary interface, increased permeability (contributing to pulmonary edema), compromised gas exchange, and hypoxemia. Diffuse alveolar damage is associated with permanent damage to the alveoli epithelial cells and capillary endothelial cells, and intracapillary thrombosis. (Batah and Fabro, Respir Med 2021 January; 176:106239). In severe cases this injury leads to a requirement for intubation and mechanical ventilation and, in some patients, particularly those with comorbidities, to death. Restoration of lung function requires regeneration of alveolar and vascular tissue such that the alveolar: capillary barrier is regenerated and efficient gas exchange is restored. Single cell analysis of cells retrieved from brochoalveolar lavage fluid of COVID19 patients has demonstrated significant expansion in cells exhibiting properties of lung stem and progenitor cells. (Zhao et al., Cell Prolif 2020 December; 53(12):e12931. doi: 10.1111/cpr.12931). One study assessed regeneration in lungs removed from COVID19 patients as part of lung transplant for severe disease. In this study the authors reported that production of new functional alveolar type I cells from their progenitors may take weeks in the context of SARS CoV2 infection (Chen et al., Cell Res. 2020 August; 30(8):708-710.). This delay is due, at least in part, to concomitant injury to the pulmonary endothelium which impairs the niche required by pulmonary stem and progenitor cells for optimal production of new epithelial cells (Ding, Hisata).

While some patients, particularly children, exhibit less severe disease and more rapid repair and restoration of normal lung function, many patients exhibit profound and sustained lung function deficits. This long-term deficit forms part of the condition referred to as Long COVID Syndrome. As with COPD, this is due, at least in part, to differences in the capacity of the patient's pulmonary repair and regeneration mechanisms to mediate repair. This delay and the ongoing persistent lung deficit present indicate a clear opportunity for regenerative approaches that accelerate lung repair and regeneration. Further, there are clear parallels between the chronic setting of long COVID syndrome and chronic progressive COPD and between the acute respiratory distress of early COVID19 and the acute exacerbation episodes of COPD.

### Pulmonary Fibrosis

In some embodiments, the lung injury treated by the methods and compositions of the present invention is associated with Idiopathic pulmonary fibrosis (IPF). IPF is one of the most frequently diagnosed interstitial lung diseases (ILDs). Although antifibrotic agents can beneficially modulate the progressive decline in lung function associated with ongoing fibrotic processes characteristic of IPF they do not promote pulmonary repair and regeneration. It is believed that IPF arises from chronic and/or repetitive microinjuries of the pulmonary epithelium resulting in activation of profibrotic processes. Pathological changes to the lung epithelium can negatively influence crosstalk between stem cells and their niche further contributing to disease progression. As with COPD, ongoing injury and persistent and prolonged activation of repair and regeneration processes lead to impaired regenerative function and stem and progenitor cell exhaustion. Hence, mechanisms that promote repair either directly on pulmonary stem and progenitor cells or on the niche within which they reside, and function have the capacity to promote repair and regeneration in pulmonary fibrosis.

### Lung Resection

In some embodiments, the lung injury treated by the methods and compositions of the present invention is that associated lung resection, i.e., surgical removal of lung tissue. Surgical removal of lung tissue is applied in several settings including, but not limited to, treatment of trauma (for example, following a thoracic gunshot wound), as part of removal or reduction of tumor burden in patients with malignant disease originating in or metastasizing to the lung, and in patients with lung disease where the deficient tissue is impairing the function of less injured or healthy lung tissue (lung volume reduction surgery). As with many organs, the body has natural compensatory mechanisms whereby reduction in volume/function leads to activation of regenerative mechanisms. Ding et al have shown that (a) this occurs in the lung in response to partial pneumonectomy, (b) that optimal repair and regeneration is dependent upon the pulmonary endothelium, and (c) that repair and regeneration can be enhanced by administration of pulmonary endothelial cells but not by non-pulmonary endothelial cells.

### Mechanism of Action

Without being limited by a particular theory, the endothelial cells disclosed herein act by one or several mechanisms, including, but not limited to directly providing a microenvironmental niche within which cells responsible for pulmonary repair and regeneration can function more effectively (that is, the cells temporarily replace or supplement the endogenous niche acting directly on tissue stem/progenitor cells); directly rescuing (in part or in total) injured pulmonary tissue endothelial cells normally responsible for maintaining the microenvironmental niche within which cells responsible for tissue repair can function more effectively from the effects of chemotherapy (that is, the endothelial cells reduce, for example, endothelial cell apoptosis/death and/or enhance endothelial recovery such that the native niche is restored more quickly); directly rescuing (in part or in total) injured pulmonary endothelial cells normally responsible for angiogenesis within the tissue without which parenchymal cell repair will be limited; by slowing loss of endothelial cell number and/or function such that the rate of reduction in endogenous endothelial cell number and/or function in response to chemotherapy is slowed such that that a higher level of residual endothelial angiogenic and/or niche function is preserved long enough for endogenous repair mechanisms to recover and directly restore tissue function.

Accordingly, provided herein are methods to replace or supplement the microenvironmental niche in a subject in need thereof, that includes administration of a composition that includes endothelial cells as described herein. Other methods relate to the rescue of the cells that comprise the niche, or of rescuing angiogenic capacity, or by slowing the loss of native pulmonary repair capacity such that repair or the repair mechanisms themselves has time to recover before the sequelae of the underlying pulmonary pathophysiology (e.g.: SARS CoV2 infection) reach the threshold needed for a clinically severe impairment of lung function.

### Methods of Treatment

Various embodiments of the methods of treatment provided by the present invention are described in the Summary of the Invention and Claims section of this patent disclosure, and in the “treatment” definition provided above. Certain additional description relating to such methods is provided here.

In some embodiments, the present invention provides methods for improving (or reducing the decline in) lung function measures such as FEV₁, FVC, inspiratory volume, elastance, functional residual capacity, diffusion capacity for CO (frequently abbreviated as DLCO), tidal volume, and total lung capacity. These parameters may be assessed as absolute values (for example in milliliters), as relative to reference values (for example: as a percentage of a healthy person of the same sex, age, and height) or as ratios (for example: FEV₁/FVC).

In some embodiments, the methods described herein do not completely prevent worsening or exacerbation of lung function but reduce the rate or magnitude of decline such that a subject so treated would experience a longer duration of higher function.

In some embodiments biological measures are used as indicators of a therapeutic effect. Examples of such biological measures have been reported in the scientific literature (for example see: Shaw et al J Thorac Dis. 2014 November; 6(11): 1532-1547 “Biomarkers of progression of chronic obstructive pulmonary disease (COPD)”) and include, but are not, limited to, C-reactive protein (CRP), interleukin 6, interleukin 8, interleukin 15, fibrinogen, tumor necrosis factor-α, the adipokine C1q/TNF-Related Protein 9 (CTRP9), volatile compounds, and cell sub-types. In some embodiments these biomarkers are measured in blood or a blood-derived sample (e.g.: plasma or serum). In other embodiments these biomarkers are measures in exhaled air or other lung-derived samples such as bronchiolar lavage fluid or a biopsy.

In some embodiments imaging measures are used as indicators of a therapeutic effect. By way of non-limiting example, it is recognized in the art that high resolution computed tomography is useful in the diagnosis, classification, and assessment of progression of several different lung diseases.

In some embodiments, the treatment involves only a single administration of the endothelial cells. In other embodiments, multiple or repeated treatment is applied. For example, in some embodiments a single administration of cells is sufficient. In other embodiments, a subject receives multiple treatments over time. Such treatments may be applied at intervals measured in days, weeks, months, or years. That is, in some embodiments repeated treatments are given over consecutive or non-consecutive days (e.g.: on two or more consecutive days, on alternating days, or with two or more days between treatments). In another embodiment, treatments is administered weekly. In other embodiments multiple treatments are administered on an ad hoc basis as indicated by the subject's disease status and response to prior treatment.

The skilled artisan will appreciate that any art-accepted technique to classify the grade, type, or progression of lung disease is useful in the embodiments disclosed herein.

### Methods of Administration

The present invention provides methods that involve administration of effective amounts of compositions comprising UVECs to subjects.

Any suitable route of administration may be used.

In some embodiments the compositions are administered to the subjects intravenously, for example by (IV) infusion. In some embodiments the IV infusion is administered peripherally, e.g., using a peripheral IV catheter. In some embodiments the IV infusion is administered centrally, e.g., using a peripherally inserted central catheter (PICC), a central venous catheter (CVC), a Hickman line, or a portacath device. In some embodiments the compositions are administered to the subject intra-arterially (for example, via the pulmonary artery or by delivery into the right ventricle or atrium). In some embodiments the compositions are delivered by retrograde infusion; for example, into the venous system draining the lungs. In other embodiments, the compositions are delivered intra-tracheally.

Any suitable timing of administration or rate of administration or administration regimen may be used.

In some embodiments the composition is administered to the subject only once, e.g., in single IV infusion.

In some embodiments the composition is administered to the subject multiple times e.g., in multiple IV infusions.

In some embodiments, the composition is administered to the subject by IV infusion over a desired period of time. In some embodiments the effective amount of the composition is administered to the subject by IV infusion over a period of about 1 min, 2 min, 3 min, 4 min, 5 min, 10 min, 30 min, 45 min, 1 h, 2 h, or longer.

In some embodiments the effective amount of the compositions is administered to the subject by IV infusion at a rate of about 6 ml/minute.

In some embodiments, the composition is administered to the subject in a first IV infusion on Day 0, a second IV infusion on about Day 28, and optionally a third IV infusion on about Day 56.

In some embodiments, wherein the subject has acute respiratory distress syndrome (e.g., associated with COVID-19) and the composition is administered to the subject in a first IV infusion on Day 0, optionally a second IV infusion on about Day 3, optionally a third IV infusion on about Day 6, and optionally a fourth IV infusion on about Day 9.

In some embodiments, the composition is administered to the subject at a dose of about 1×10⁶ cells per kg bodyweight, per infusion.

In some embodiments the composition is administered to the subject at a dose of about 2.5×10⁶ cells per kg bodyweight, per infusion.

In some embodiments, the composition is administered to the subject at a dose of about 5×10⁶ cells per kg bodyweight, per infusion.

In some embodiments, the composition is administered to the subject at a dose of about 10×10⁶ cells per kg bodyweight, per infusion.

In some embodiments, the composition is administered to the subject at a dose of about 20×10⁶ cells per kg bodyweight, per infusion.

In some embodiments the composition is administered to the subject at a dose of between 1 million cells per kilogram of subject body weight and 500 million cells per kilogram, per infusion.

In some embodiments the composition is administered to the subject at a dose of between approximately 1 and 50 million cells per kilogram of subject body weight, per infusion.

In some embodiments the composition is administered to the subject at a dose of between approximately 1 and 20 million cells per kilogram of subject body weight, per infusion.

In some embodiments the composition is administered to the subject at a dose of between approximately 1 and 20 million cells per kilogram of subject body weight, per infusion.

In some embodiments the composition is administered to the subject at a dose of between approximately 1 and 10 million cells per kilogram of subject body weight, per infusion.

In some embodiments the composition is administered to the subject at a dose of between approximately 2.5 and 20 million cells per kilogram of subject body weight, per infusion.

In some embodiments the composition is administered to the subject at a dose of between approximately 2.5 and 10 million cells per kilogram of subject body weight, per infusion.

In some embodiments the composition is administered to the subject at a dose of between approximately 2.5 and 5 million cells per kilogram of subject body weight, per infusion.

In some embodiments the composition is administered to the subject at a dose of between approximately 5 and 20 million cells per kilogram of subject body weight, per infusion.

In some embodiments the composition is administered to the subject at a dose of between approximately 5 and 10 million cells per kilogram of subject body weight, per infusion.

In some embodiments the composition is administered to the subject at a dose of between approximately 10 and 20 million cells per kilogram of subject body weight, per infusion.

In some embodiments the composition is administered to the subject at a dose of at least 20 million cells per kilogram of subject body weight, per infusion.

In some embodiments, where multiple administrations are employed, the first administration of the composition may be at one dose and subsequent administrations may be at a different dose or doses.

In some embodiments, where multiple administrations are employed, the first administration of the composition may be via one route and subsequent administrations may be by another route. By way of example only, in some embodiments administration at one time might be in the form of infusion into a peripheral vein, whereas at another time the administration may be in the form of infusion into the pulmonary artery.

### Endothelial Cells

The methods and compositions of the present invention involve umbilical vein endothelial cells (UVECs). UVECs are isolated from umbilical cords. The endothelial cells disclosed herein can be isolated from the umbilical cord using any means known to those skilled in the art.

By way of example only, UVECs can be isolated from umbilical vein tissue by a process wherein tissue is excised and mechanically broken into fragments. Enzymes may also be perfused through the major vessels of the umbilical cord from to release the endothelial cells lining the interior of the vessels and any vessels downstream thereof. Once released, the cells can be suspended and cultured in a medium supportive of endothelial cell growth. In some embodiments the cell population so isolated can be further purified, for example, by immunoselection of endothelial cells using a marker or combination of markers that is selective for endothelial cells (for example, antibody directed to CD31). The endothelial cells may be expanded in culture. In one embodiment the endothelial cells are transfected with a gene conferring a growth advantage over non-transduced cells such that transduced cells grow more rapidly and/or more stably than the non-transduced cells from which they were derived.

Additional methods for preparation of UVECs are provided in the Examples section of this patent disclosure.

In some embodiments the umbilical cord endothelial cells (UVECSs) are genetically modified.

In preferred embodiments the UVECs are genetically modified to express the adenovirus E4ORF1 protein, i.e., they are E4ORF1+UVECs.

In some embodiments, the UVECsls are engineered to express one or more ETS family transcription factors, (i.e. they are ETS+UVECs). In some embodiments, the UVECs are engineered to express the ETS family transcription factor ETV2 (i.e. they are ETV2+UVECs). In some embodiments, the UVECs are E4ORF1+ETV2+UVECs.

### E4ORF1

Several of the embodiments of the present invention involve UVECs that are E4ORF1+—i.e., UVECs that express an E4ORF 1 polypeptide. The “E4ORF1” polypeptide is encoded by open reading frame (ORF) 1 of the early 4 (E4) region of the adenovirus genome. E4ORF1+UVECs for use in accordance with the present invention typically comprise a recombinant nucleic acid molecule that contains an E4ORF1 coding sequence operatively linked to a promoter suitable for expression of the E4ORF1 coding sequence in UVECs.

E4ORF1 amino acid sequences and nucleotide sequences are known in the art. Any such sequences may be used in accordance with the present invention. In some embodiments the E4ORF1 polypeptide may be from any suitable adenovirus type or strain, such as human adenovirus type 2, 3, 5, 7, 9, 11, 12, 14, 34, 35, 46, 50, or 52. In some preferred embodiments the polypeptide sequence used is from human adenovirus type 5. Amino acid sequences of such adenovirus polypeptides, and nucleic acid sequences that encode such polypeptides, are well known in the art and available in well-known publicly available databases, such as the Genbank database. For example, suitable sequences include the following: human adenovirus 9 (Genbank Accession No. CAI05991), human adenovirus 7 (Genbank Accession No. AAR89977), human adenovirus 46 (Genbank Accession No. AAX70946), human adenovirus 52 (Genbank Accession No. ABK35065), human adenovirus 34 (Genbank Accession No. AAW33508), human adenovirus 14 (Genbank Accession No. AAW33146), human adenovirus 50 (Genbank Accession No. AAW33554), human adenovirus 2 (Genbank Accession No. AP.sub.—000196), human adenovirus 12 (Genbank Accession No. AP.sub.—000141), human adenovirus 35 (Genbank Accession No. AP.sub.—000607), human adenovirus 7 (Genbank Accession No. AP.sub.--000570), human adenovirus 1 (Genbank Accession No. AP.sub.—000533), human adenovirus 11 (Genbank Accession No. AP.sub.—000474), human adenovirus 3 (Genbank Accession No. ABB 17792), and human adenovirus type 5 (Genbank accession number D12587). In one embodiment the E4ORF1 sequence used is that having NCBI accession number AZR66741.1. In one embodiment the E4ORF1 sequence used is that having NCBI accession number AP 000232.1.

In one embodiment the E4ORF1 sequence used is has the amino acid sequence:

(SEQ ID NO. 1)
MAAAVEALFVVLEREGAILPRQEGFSGVYVFFSPINFVIPPMGAVMLSL
RLRVCIPPGYFGRFLALTDVNQPDVFTESYIMTPDMTEELSVVLFNHGD
QFFYGHAGMAVVRLMLIRVVFPVVRQASNV.

In some embodiments the E4ORF1 polypeptide may have amino acid sequence that is, or is encoded by a nucleic acid sequence that is, a variant, derivative, mutant, or fragment of any of the specific sequences provided herein or known in the art provided that such variants, derivatives, mutants, or fragments are, or encode, a polypeptide that has one or more of the functional properties of adenovirus E4ORF1 described herein. In some embodiments, the variants, derivatives, mutants, or fragments have about an 85% identity to the known sequence, or about an 88%, 89%, 90%, 91%, 92%, 93%, 94%, 95%, 96%, 97%, 98%, or 99% sequence identity to the known sequence. In some embodiments, a variant, derivative, mutant, or fragment of a known nucleotide sequence is used that varies in length by about 50 nucleotides, or about 45 nucleotides, or about 40 nucleotides, or about 35 nucleotides, or about 30 nucleotides, or about 28 nucleotides, 26 nucleotides, 24 nucleotides, 22 nucleotides, 20 nucleotides, 18 nucleotides, 16 nucleotides, 14 nucleotides, 12 nucleotides, 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide relative to the known nucleotide sequence. In some embodiments, a variant, derivative, mutant, or fragment of a known amino sequence is used that varies in length about 50 amino acids, or about 45 amino acids, or about 40 amino acids, or about 35 amino acids, or about 30 amino acids, or about 28 amino acids, 26 amino acids, 24 amino acids, 22 amino acids, 20 amino acids, 18 amino acids, 16 amino acids, 14 amino acids, 12 amino acids, 10 amino acids, 9 amino acids, 8 amino acids, 7 amino acids, 6 amino acids, 5 amino acids, 4 amino acids, 3 amino acids, 2 amino acids, or 1 amino acid relative to the known amino acid sequence.

In some embodiments E4ORF1 sequences are used without other sequences from the adenovirus E4 region—for example not in the context of the entire E4 region or not together with other ORFs in the E4 region. However, in other embodiments E4ORF1 may be used in conjunction with one or more other ORFs from the E4 region, such as E4ORF2, E4ORF3, E4ORF4, E4ORF5 or E4ORF6/7 sequences. For example, although E4ORF1 sequences can be used in constructs (such as a viral vectors) that contain other sequences, genes, or coding regions (such as promoters, marker genes, antibiotic resistance genes, and the like), in certain embodiments, the E4ORF1 sequences are used in constructs that do not contain the entire E4 region, or that do not contain other ORFs from the entire E4 region, such as E4ORF2, E4ORF3, E4ORF4, and/or E4ORF5.

E4ORF1 encoding sequences can be present in constructs or vectors that contain various other sequences, genes, or coding regions, for example, promoters, enhancers, antibiotic resistance genes, reporter genes or expression tags (such as, for example nucleotides sequences encoding GFP), or any other nucleotide sequences or genes that might be desirable.

E4ORF1-encoding nucleic acid molecules can be under the control of one or more promoters to allow for expression. Any promoter able to drive expression of the E4ORF1 nucleic acid sequences in endothelial cells can be used. Examples of suitable promoters include, but are not limited to, the CMV, SV40, RSV, HIV-Ltr, and MML promoters. The promoter can also be a promoter from the adenovirus genome, or a variant thereof. For example, in some embodiments the promoter may be a promoter that drives expression of E4ORF 1 in nature in an adenovirus genome. However, in other embodiments the promoter is not one that drives expression of E4ORF1 in nature in an adenovirus genome.

The E4ORF1-encoding sequences may comprise naturally occurring nucleotides, synthetic nucleotides, or a combination thereof. For example, in some embodiments the nucleic acid molecules of the invention can comprise RNA, such as synthetic modified RNA that is stable within cells and can be used to direct protein expression/production directly within cells. In other embodiments the E4ORF1-encoding sequences can comprise DNA. In embodiments where DNA is used, the DNA sequences may be operably linked to one or more suitable promoters and/or regulatory elements to allow (and/or facilitate, enhance, or regulate) expression within cells, and may be present in one or more suitable vectors or constructs.

The E4ORF1-encoding sequences can be introduced into UVECs using any suitable system known in the art, including, but not limited to, transfection techniques and viral-mediated transduction techniques. Transfection methods that can be used in accordance with the present invention include, but are not limited to, liposome-mediated transfection, polybrene-mediated transfection, DEAE dextran-mediated transfection, electroporation, calcium phosphate precipitation, microinjection, and micro-particle bombardment. Viral-mediated transduction methods that can be used include, but are not limited to, lentivirus-mediated transduction, adenovirus-mediated transduction, retrovirus-mediated transduction, adeno-associated virus-mediated transduction and herpesvirus-mediated transduction.

In some embodiments the E4ORF1-encoding sequences are in a vector. In some embodiments the E4ORF1-encoding sequences are in a viral vector. In some embodiments the E4ORF1-encoding sequences are in a lentiviral vector. In some embodiments the E4ORF1-encoding sequences are in an adenoviral vector. In some embodiments the E4ORF1-encoding sequences are in adeno-associated virus vector. In some embodiments the E4ORF1-encoding sequences are in a retroviral vector. In some embodiments the E4ORF1-encoding sequences are in a Moloney murine leukemia virus (MMLV) vector (a type of retroviral vector).

In some embodiments the presence of E4ORF1 coding sequences in the UVECs can be confirmed and/or quantified using standard nucleic acid detection and/or quantification assays known in the art, such as PCR-based techniques (e.g., quantitative PCR) and sequencing-based techniques (e.g., quantitative next generation sequencing-based techniques). In some embodiments the presence of E4ORF1 polypeptides can be confirmed and/or quantified using standard protein detection and/or quantification assays known in the art, such as antibody-based techniques. In some embodiments the expression of functional E4ORF1 polypeptide (or an appropriate amount of functional E4ORF1 polypeptide can be confirmed and/or quantified using functional assays (e.g., in vitro or in vivo assays) for any of the functional properties of E4ORF1-expressing endothelial cells that are known in the art (such as any of those described in U.S. Pat. No. 8,465,732).

The handling, manipulation, and expression of E4ORF1 sequences in UVECs may be performed using conventional techniques of molecular biology and cell biology. Such techniques are well known in the art. For example, one may refer to the teachings of Sambrook, Fritsch and Maniatis eds., “Molecular Cloning A Laboratory Manual, 2nd Ed., Cold Springs Harbor Laboratory Press, 1989); the series Methods of Enzymology (Academic Press, Inc.), or any other standard texts for guidance on suitable techniques to use in handling, manipulating, and expressing nucleotide and/or amino acid sequences. Additional aspects relevant to the handling and expression of E4ORF1 sequences in endothelial cells are described in U.S. Pat. No. 8,465,732, the contents of which are hereby incorporated by reference.

### Compositions

In some embodiments the present invention provides compositions comprising an effective amount of UVECs (e.g., E4ORF1+ HUVECs). Typically, such compositions comprise UVECs in a physiological saline solution, such as a physiological saline suitable for administration to a subject. In some embodiments the compositions may comprise one or more additional excipients suitable for administration to subjects and compatible with cell therapies, such as buffers, salts, preservatives, polysaccharides (e.g., dextrans), proteins (e.g., albumin) and the like. In some embodiments the compositions may comprise a low concentration of a cryopreservative. In some embodiments the compositions of the present invention comprise Dextran 40. In some embodiments the compositions of the present invention comprise human serum albumin (HSA). In some embodiments the compositions of the present invention comprise dimethyl sulfoxide (DMSO), preferably at a concentration of up to 0.25%.

In some embodiments the compositions of the present invention comprise E4ORF1+ HUVEC cells, Dextran40, HSA and DMSO in a physiological saline. In some such embodiments such compositions comprise E4ORF1+ HUVEC cells at a concentration of about 5×10⁶ cells per ml. In some such embodiments such compositions comprise Dextran 40 at a concentration of about 8 mg/ml. In some such embodiments such compositions comprise HSA at a concentration of about 4.5 mg/ml. In some such embodiments such compositions comprise DMSO at a concentration of about 0.25 mg/ml.

In some embodiments the compositions of the present invention comprise both E4ORF1+ and E4ORF1-negative UVECs. In some embodiments at least about 75% of the UVECs in the compositions are E4ORF1+. In some embodiments at least about 80% of the UVECs in the compositions are E4ORF1+. In some embodiments at least about 85% of the UVECs in the compositions are E4ORF1+. In some embodiments at least about 90% of the UVECs in the compositions are E4ORF1+. In some embodiments at least about 95% of the UVECs in the compositions are E4ORF1+. In some embodiments at least about 98% of the UVECs in the compositions are E4ORF1+. In some embodiments at least about 99% of the UVECs in the compositions are E4ORF1+.

Typically, the UVECs (e.g., E4ORF1+ HUVECs) for use in the methods of the present invention are supplied (e.g., to the medical facility where there will be administered to subjects) in frozen form—i.e., in a frozen composition containing the specified endothelial cells (e.g., E4ORF1+ HUVECs) and a cryopreservative. The frozen compositions are thawed prior to use, for example using standard methods known in the art. In some embodiments the frozen composition may be thawed and then administered to a subject without dilution. However, in other embodiments the frozen composition is thawed and then diluted in a suitable diluent to result in the generation of a composition containing the desired or specified concentration cells and the desired or specified concentrations of excipients, for example as described above.

In some embodiments the present invention provides a kit comprising a composition according to the present invention in sterile packaging and instructions for administering the therapeutic composition to a subject for treatment of lung injury.

### Combination Therapy

In some embodiments the methods of treatment provided herein can be combined with other treatment methods, e.g., by administering to the subjects one or more additional agents (e.g., pharmaceutical agents, biologic agents, or cell therapy agents, other therapeutic agents) in addition to the UVEC-containing compositions described herein.

In some embodiments, the one or more additional agents can be administered in a single composition together with the UVECs (i.e., the additional agents can be added to the compositions of the present invention). In some embodiments, one or more additional agents can be administered separately from the compositions of the present invention. For example, in some embodiments, one or more additional agents can be administered just prior to, or just after, administration of the UVEC-containing compositions of the present invention. As used herein, the term “just prior” can refer to within 15 minutes, 30 minutes, an hour, 2 hours, 3 hours, 4 hours, 5 hours, or the like. Likewise, the phrase “just after administration” can refer to within 15 minutes, 30 minutes, an hour, 2 hours, 3 hours, 4 hours, 5 hours, or the like.

In some embodiments the treatment is administered along with an immunosuppressive agent or immunosuppressive regimen (for example: anti-thymocyte globulin, mycophenylate mofetil, cyclosporin, methotrexate, cyclophosphamide, Rituxan, steroidal agent, or combinations thereof). In some such embodiments, administration of the immunosuppressive agent or regimen is initiated up to 10 days prior to administration of the treatment. In other embodiments, administration of the immunosuppressive agent or regimen is initiated up to 10 days following administration of the treatment. In some embodiments, administration of the immunosuppressive agent or regimen is initiated simultaneously with administration of treatment.

Additional agents useful in combination therapy in the methods described herein include, for example, growth factors, cytokines, anti-inflammatory agents, anti-bacterial, anti-viral, and anti-fungal agents, as well as other agents known in the art to have beneficial effects on, for example, lung injury, emphysema, airway constriction, and/or vasoconstriction.

The invention is further described with reference to the following non-limiting Examples.

### EXAMPLES

The following examples are provided to demonstrate particular situations and settings in which this technology may be applied and are not intended to restrict the scope of the invention and the claims included in this disclosure.

### Example 1

### Preparation of E4ORF1+ HUVECs

HUVECs were isolated from the umbilical vein of an umbilical cord by enzymatic digestion with collagenase. The endothelial cells were then cultured in growth medium composed of M199 base medium supplemented with 10% fetal bovine serum, 20 ng/ml FGF-2, 10 U/mL heparin, 40 μg/mL Gentamicin, 10 mM HEPES and 1× Glutamax.

After 2 days in culture, HUVECs were transduced with a retroviral vector containing an E4ORF1 expression cassette (encoding SEQ ID NO.1) and a gentamicin resistance gene. After culturing for several days, the cells were switched to culture in selection medium (containing gentamicin) to select for transduced cells. The resulting E4ORF1+ HUVEC cells are referred to throughout these Examples and the Figures referenced therein as “E-CELs” or “E-CEL UVECs or “E-CEL UVEC cells.”

### Example 2

### Preclinical Studies: Lung Function

Eight-week-old male C57Bl/6 mice underwent intratracheal instillation of elastase using a borescope with video camera. Animals received a single dose of elastase in a concentration set to require a volume of 50 μL/25 g BW (body weight) to deliver desired dose of 2 U/25 g BW. Animals assigned to the uninjured control group received instillation of saline rather than elastase. Because of difficulty in reproducibly infusing elastase into the distal lung, an injury, an acceptance criterion was applied in which animals with low injury (as evidenced by a decline of less than 1.5% of body weight) were not included in the study. This model is very similar to that applied by Hisata et al.

E-CEL UVEC cells were administered intravenously (via the retro-orbital sinus) at a dose of 1,000,000 cells in a volume of 0.1 mL. In one study the first E-CEL UVEC administration was performed in the acute phase of injury (one week after elastase instillation). In this study, additional infusions were performed on day 9 and 11 after injury. In another study, the first E-CEL UVEC administration was performed in the chronic phase of injury (30 days after elastase instillation).

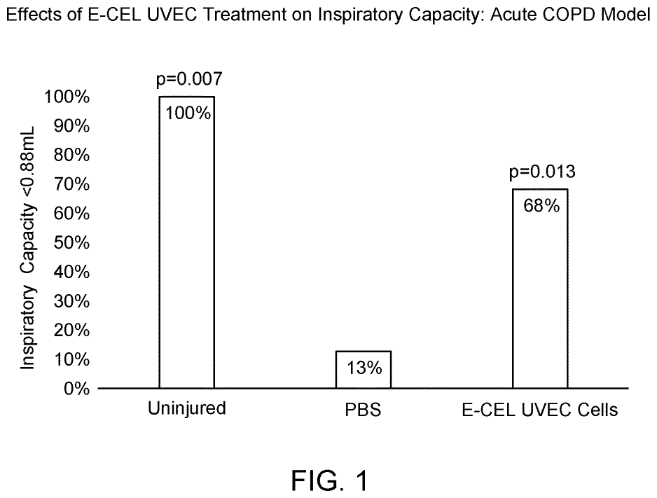

The results shown in FIG. 1 showed than elastase injury resulted in substantial impairment in lung function such that, whereas 100% of uninjured animals exhibited inspiratory capacity <0.88 mL, this was evident in only 13% of injured, untreated animals (p=0.007). By contrast, a significantly higher percentage (68%) of animals treated with E-CEL UVEC cells exhibited superior inspiratory capacity (p=0.013; compared to animals not receiving E-CEL UVEC cells). The same pattern was evident when the end point assessed was elastance, a measure of the elastic properties of the lung. 100% of uninjured animals exhibited elastance >34 at a pressure of 30 cm H₂O compared to only 13% of injured: untreated animals (p=0.005) and 68% of E-CEL UVEC cell-treated animals (p=0.013 for comparison to untreated animals).

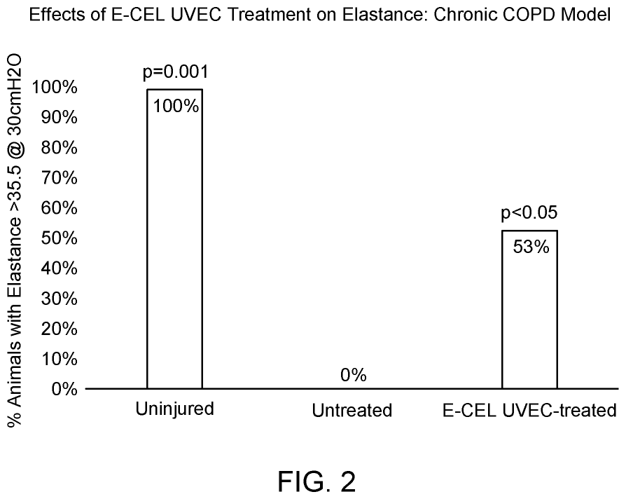

Essentially identical trends were evident when E-CEL UVEC cell delivery was delayed until the chronic phase of injury (day 28 after injury) (FIG. 2).

Both of these studies demonstrate the capacity of E4ORF1+ HUVECs to improve lung function in a relevant animal model despite the fact that the endothelial cells are not of pulmonary tissue origin.

### Example 3

### Preclinical Studies: Lung Endothelial Cell Repopulation

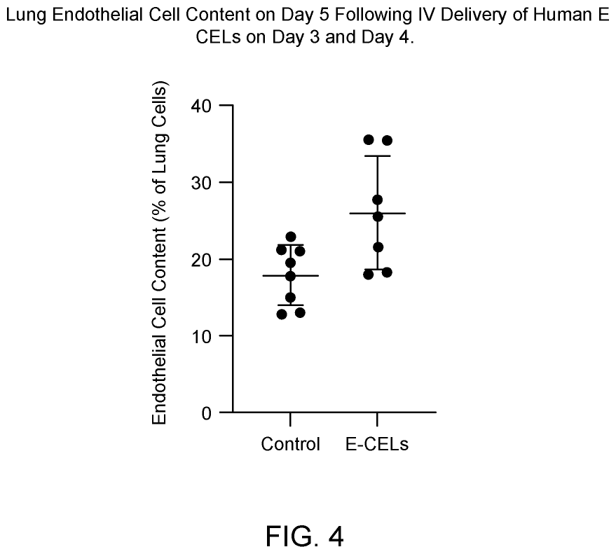

Hisata et al. reported that elastase injury led to decrease in lung endothelial cell content (Hisata et al., FIG. 2E) and that treatment with E4ORF1-expressing mouse lung endothelial cells led to accelerated endothelial repopulation with increased endothelial cell frequency (Hisata et al., FIG. 4E) evident following treatment with lung endothelial cells but not lung fibroblasts or endothelial cells from a tissue other than lung (adipose). Hisata et al., further reported increased mouse lung endothelial cell proliferation on Day 10 and Day 17 after treatment with mouse lung endothelial cells (Hisata et al., FIG. 4I).

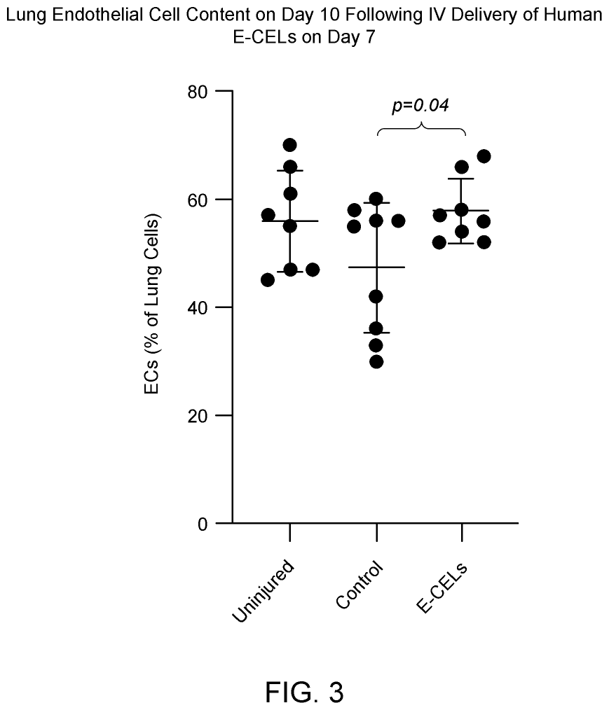

Using the same injury model and timing of endothelial cell administration (Day 7 and Day 14 after injury) we found increased mouse lung endothelial cell content in animals treated with human E-CELs (FIG. 3). Mean total endothelial cell number in the lungs of E-CEL treated animals was also greater than that in the untreated controls (42.0±9.2×10³ cells versus 33.3±8.0×10³ cells). Assessment of mouse lung endothelial cell number on Day 19 after injury following the Hisata et al., delivery schedule (Day 7 and Day 14) showed that mean total endothelial cell number in the lungs of E-CEL treated animals was greater than that in the untreated controls (30.3±5.4×10³ cells versus 24.6±2.9×10³ cells). This difference was statistically significant (p=0.019).

Thus, contrary to Hisata et al., where mouse lung endothelial cell repopulation was mediated only by mouse lunge endothelial cells, these data show that human E-CELs mediate a similar response.

In the same model, we also found that delivery of human E-CELs on Day 3 and Day 4 after injury led to a significant increase in mouse pulmonary endothelial cells on Day 5 (FIG. 4).

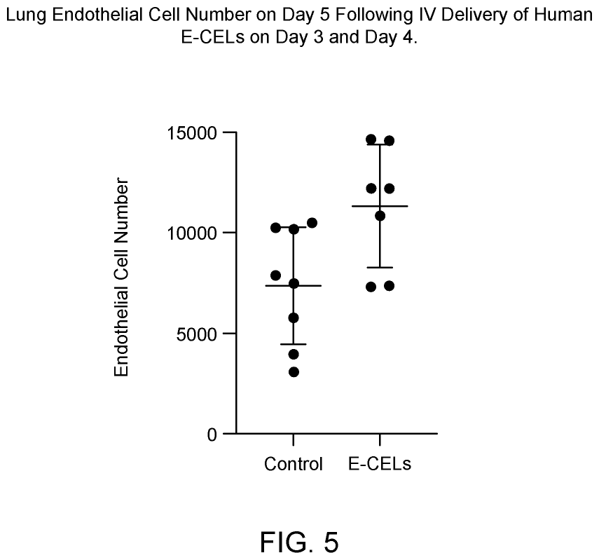

Mean endothelial cell number on Day 5 was significantly higher in the E-CEL treated cohort compared to the control animals (11.3+3.0×10³ cells versus 7.4=2.9×10³ cells; p=0.02; FIG. 5).

### Example 4

### Preclinical Studies: Lung Bronchoalveolar Stem Cell Repopulation

Ding et al., reported that that mouse lung endothelial cells promote proliferation of bronchoalveolar stem cells (BASCs) in a tissue-specific manner (in that mouse liver endothelial cells did not mediate this effect). Contrary to this teaching, we have discovered that human E-CELs are capable of stimulating mouse BASCs.

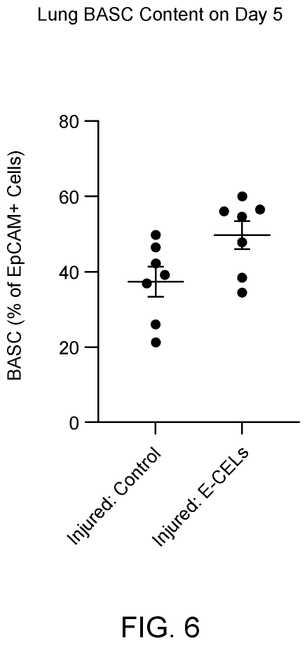

Using the same elastase model, we found that intravenous delivery of 500,000 human E-CEL UVEC cells on Day 2 and Day 3 following instillation of elastase led to a statistically significant increase in the frequency of mouse bronchoalveolar stem cells (BASCs) in the lungs on Day 5. Mean total BASC number in the lungs of E-CEL treated animals was numerically greater than that in the untreated controls on Day 5 (5.6±4.0×10³ cells versus 4.1±2.6×10³ cells. Specifically, mean BASC content (EpCAM⁺/Sca-1⁺ cells as a percentage of lung epithelial cells) in E-CEL UVEC cell-treated animals was significantly greater than that in the untreated controls (49.7±9.8% versus 37.4±10.4%; p=0.042; FIG. 6). No deleterious reactions were noted in animals treated with E-CEL UVEC cell administration.

These data clearly demonstrate that UVECs promote lung regeneration; a finding that is unexpected given the requirement for tissue-specificity reported by others previously, including by Hisata et al., when using the same injury model.

### Example 5

### Preclinical Studies: Lung Fibrosis

Six-eight-week-old male C57Bl/6 mice underwent intranasal instillation of bleomycin (4U/kg; 2U/mL in 0.9% saline; 50 μL/mouse). Animals assigned to the uninjured control group received instillation of saline rather than bleomycin. This model is very similar to that applied by Lee et al., who reported that regeneration in response to this injury was mediated by medium conditioned by lung endothelial cells but not by medium from liver endothelial cells.

E-CEL UVEC cells were administered intravenously (via the tail vein) at a dose of 500,000 cells (corresponding to ~20×10⁶/kg) in a volume of 0.2 mL 1, 6, and 11 days after injury. The outcome was assessed using histologic assessment of the lung for fibrosis (Fibrotic Index), inflammation, and hemorrhage at day 19 after injury. Fibrosis was scored using the Modified Ashcroft scoring system. Ten 20× fields from each mouse were scored individually on a scale of 0-8 and the mean calculated to generate a fibrosis index score. Inflammation and hemorrhage were scored from 0-5 where 0 is normal, 1 is minimal change, 2 is mild change, 3 is moderate change, 4 is marked change, and 5 is severe change.

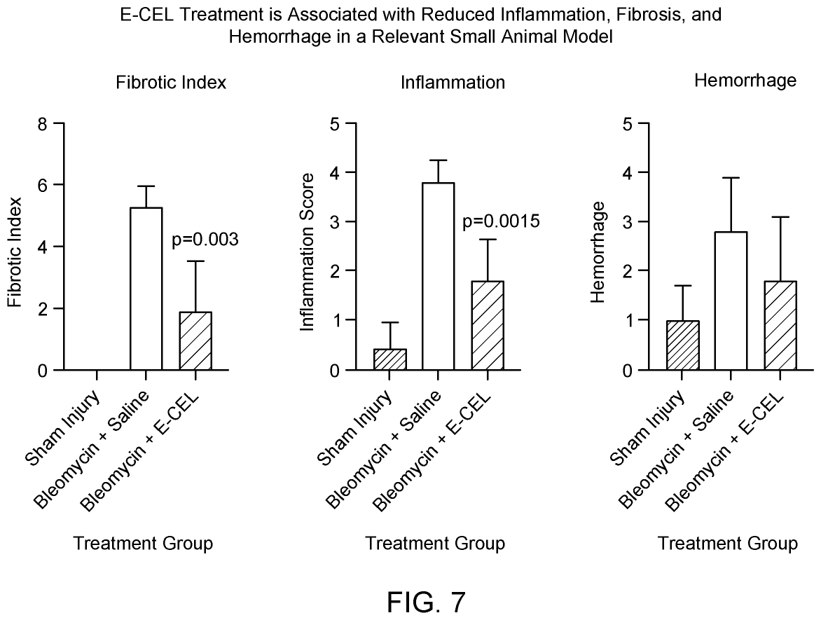

The data shown in FIG. 7 shows that E-CEL treatment was associated with statistically significantly lower fibrotic index and inflammation score and a numerically lower hemorrhage score at day 19 after injury.

These data, obtained using a second lung injury model, clearly demonstrate that UVECs are useful in treatment of lung injury; a finding that is unexpected given the requirement for tissue-specificity reported by Lee et al., when using the same injury model.

### Example 6

### Clinical Trial in COPD

A synopsis of key clinical trial protocol parameters is listed in Table 1, below:

TABLE 1

| | |
|---|---|
| Title | A Phase I/II, Ulticenter, Randomized, Double-Blind, Placebo-Controlled |
| | Study To Evaluate The Safety And Efficacy Of Intravenous Infusion Of |
| | E4ORF1-Transduced Allogeneic Human Umbilical Vein Endothelial Cells |
| | For The Treatment Of Subjects With Mild-To-Moderate (GOLD I And II) |
| | Emphysematous Chronic Obstructive Pulmonary Disease (COPD) |
| Test Product | E4ORF1-transduced allogeneic human umbilical vein endothelial cells (E- |
| | CELs) |
| Objective | To investigate the safety of allogeneic E-CELs administered intravenously to |
| | subjects with mild-to-moderate (GOLD 1 & 2) emphysematous COPD and to |
| | assess a dose response |
| Design | Up to 5 sequential dose escalation cohorts |
| | Cohorts will be enrolled sequentially. Within each cohort subjects will be |
| | randomized 4:1 to receive either E-CELs or placebo. E-CEL doses will be as |
| | follows: |
| | Cohort 1: 1 × 10⁶/kg body weight |
| | Cohort 2: 2.5 × 10⁶/kg body weight |
| | Cohort 3: 5 × 10⁶/kg body weight |
| | Cohort 4: 10 × 10⁶/kg body weight |
| | Cohort 5: 20 × 10⁶/kg body weight |
| | Safety data will be assessed by an unblinded DRC after the last subject has |
| | reached W4 before enrollment into subsequent cohorts |
| Sample Size | 96 |
| Indication | Mild-to-moderate (GOLD 1 & 2) emphysematous chronic obstructive |
| | pulmonary disease (COPD) |
| Visit | Screening (Day −28 to Day −1), Procedure Day (Day 1), and follow up visits |
| Schedule | at weeks 1, 4, 12, 24, 36, and 48 weeks |
| Treatment | Subjects will receive one of the four doses of E-CELs specified above |
| Arms | administered by intravenous administration. Note: later stage trials including |
| | registration trials will apply a single dose level but multiple doses thereof. |
| Primary | Change over baseline in Forced Expiratory Volume (FEV1) at 24 weeks |
| Endpoint | (Note: registration trials will apply an endpoint of ≥48 weeks) |
| Secondary | Change in FEV1 at Weeks 4, 12, 24 and 36 |
| Endpoints | Number of Participants with Adverse Events (AEs) |
| | Pulmonary function tests (each assessed independently): FVC, FEV (other |
| | than 48 weeks), FEV/FVC, FRC, TLC, IC, ERV, RV, DLCO, Airway |
| | Resistance |
| | Six minute walk distance |
| | Time to acute exacerbation event |
| | Number of acute exacerbation events within 48 weeks |
| | Quantitative emphysema score on High resolution computed tomography of |
| | the lung |
| | LVEF |
| | St. George's Respiratory Questionnaire |
| | Patient reported dyspnea e.g. per Baseline and Transition Dyspnoea Index |
| | (BDI/TDI) |
| | Physician Global Assessment Scale |
| | Time to COPD Exacerbation |
| | EQ-5D |
| Biomarkers | Summary of macrophages, neutrophils, eosinophils and bronchial epithelial |
| | cells in induced sputum |
| | Plasma surfactant protein D |
| | Inflammatory biomarkers Fibrinogen, IL6, IL8, TNFa |
| Safety | Adverse events, Serious Adverse Events, |
| Inclusion & | Inclusion Criteria |
| Exclusion | Males or females ≥40 and ≤80 years of age or ≥60 years old |
| Criteria | Able to provide written informed consent |
| | Diagnosis of mild-to-moderate (GOLD 1 & 2) emphysematous chronic |
| | obstructive pulmonary disease (COPD) |
| | Pulmonary Function: FVC < 80% and >30% predicted, FEV₁/FVC < 0.7, |
| | DLCO < 80% predicted |
| | post-albuterol/salbutamol FEV1 of less than or equal to 70% of predicted |
| | Current or ex-smoker, with a cigarette smoking history of ≥10 years or >10 |
| | pack-years |
| | No change in medications for COPD in the 60 days prior to the Screening |
| | Visit. |
| | Not actively considered for lung transplant |
| | Exclusion Criteria |
| | Requirement for supplemental oxygen at rest |
| | Inability to perform a six minute walk test |
| | Diagnosis of asthma or other clinically relevant lung disease other than COPD |
| | (e.g. restrictive lung diseases, sarcoidosis, tuberculosis, idiopathic pulmonary |
| | fibrosis, bronchiectasis, or lung cancer) |
| | Subject has a body mass greater than 150 kg (330 lb) or less than 40 kg (88 |
| | lb) |
| | Subject has active infection |
| | Subject has had a significant exacerbation of COPD or has required |
| | mechanical ventilation within 4 weeks of screening |
| | Subject with clinically relevant uncontrolled medical condition not associated |
| | with COPD |
| | Subject has documented history of uncontrolled heart failure |
| | Subject has pulmonary hypertension due to left heart condition |
| | Subject has atrial fibrillation or significant congenital heart defect/disease |
| | Subject has initiated pulmonary rehabilitation within 3 months of screening |
| | Cancer requiring chemotherapy or resection within the 5 years prior to the |
| | Screening Visit (other than basal cell carcinoma) |
| | Biologic medications in the 90 days prior to the Screening Visit |
| | Known history of HIV, or has active Hepatitis B or active Hepatitis C |
| | Pregnant or lactating status. Pregnancy as determined by a positive pregnancy |
| | test prior to procedure |
| | Clinically significant abnormal findings on laboratory screening panels, |
| | including hemoglobin ≤10 g/dL or WBC ≤3 × 10⁹ cells/L |
| | Hepatic dysfunction, as defined as aspartate aminotransferase (AST), alanine |
| | aminotranferase (ALT), or bilirubin levels >1.5 times the upper limit of |
| | normal range (×ULN) prior to randomization |
| | Chronic renal insufficiency as defined as a serum creatinine > 2.0 mg/dL or |
| | requires dialysis |
| | Organ or stem cell transplantation |
| | Any clinically significant finding that would place the patient at health risk, |
| | impact the study, or affect the completion of the study |
| | Any psychiatric illness that would prevent comprehension of the details and |
| | nature of the study and interfere with follow-up clinic visits |
| | Participation in any experimental drug or device study within the 90 days |
| | prior to the Screening Visit |

### Example 7

### Clinical Trial in AECOPD

A synopsis of key clinical trial protocol parameters is listed in Table 2, below:

TABLE 2

| | |
|---|---|
| Title | A Phase I/II, Multicenter, Randomized, Double-blind, Placebo-controlled |
| | Study to Evaluate the Safety and Efficacy of Intravenous Infusion of |
| | (E4ORF1-transduced human umbilical vein endothelial cells) for the |
| | Treatment of Subjects With Acute Exacerbation of Moderate to Severe |
| | Chronic Obstructive Pulmonary Disease (COPD) |
| Test Product | E4ORF1-transduced allogeneic human umbilical vein endothelial cells (E- |
| | CELs) |
| Objective | To investigate the safety of E-CELs administered intravenously to subjects |
| | with acute exacerbation of moderate to severe COPD and to assess a dose |
| | response |
| Design | Up to 5 sequential dose escalation cohorts |
| | Cohorts will be enrolled sequentially. Within each cohort subjects will be |
| | randomized 4:1 to receive either E-CELs or placebo. E-CEL doses will be |
| | as follows: |
| | Cohort 1: 1 × 10⁶/kg body weight |
| | Cohort 2: 2.5 × 10⁶/kg body weight |
| | Cohort 3: 5 × 10⁶/kg body weight |
| | Cohort 4: 10 × 10⁶/kg body weight |
| | Cohort 5: 20 × 10⁶/kg body weight |
| | Safety data will be assessed by an unblinded DRC after the last subject has |
| | reached W4 before enrollment into subsequent cohorts |
| Sample Size | 96 |
| Indication | Acute Exacerbation of Moderate to Severe Chronic Obstructive Pulmonary |
| | Disease (COPD) |
| Visit | Screening (Day −5 to Day −1), Procedure Day (Day 1), and follow up visits |
| Schedule | at weeks 1, 4, 12, 24, 36, and 48 weeks |
| Treatment | Subjects will receive one of the four doses of E-CELs specified above |
| Arms | administered by intravenous administration. Note: later stage trials |
| | including registration trials will apply a single dose level but may apply |
| | multiple doses thereof. |
| Safety | Assessment of adverse events |
| Primary | Change over baseline in Forced Expiratory Volume (FEV1) at 24 weeks |
| Endpoint | (Note: registration trials will apply an endpoint of ≥48 weeks) |
| Secondary | Change in FEV1 at Weeks 4, 12, 24 and 36 |
| Endpoints | Number of Participants with Adverse Events (AEs) |
| | Time to next acute exacerbation event |
| | Pulmonary function tests (each assessed independently): FVC, FEV (other |
| | than 48 weeks), FEV/FVC, FRC, TLC, IC, ERV, RV, DLCO |
| | Airway Resistance |
| | Six minute walk distance |
| | Quantitative High resolution computed tomography of the lung |
| | LVEF |
| | St. George's Respiratory Questionnaire |
| | Patient reported dyspnea e.g. per Baseline and Transition Dyspnoea Index |
| | (BDI/TDI) |
| | Physician Global Assessment Scale |
| | Time to COPD Exacerbation |
| | EQ-5D |
| Biomarkers | Summary of macrophages, neutrophils, eosinophils and bronchial epithelial |
| | cells in induced sputum |
| | Plasma surfactant protein D |
| | Inflammatory biomarkers Fibrinogen, IL6, IL8, IL15, TNFa |
| Safety | Adverse events, Serious Adverse Events, |
| Inclusion & | Inclusion Criteria |
| Exclusion | Males or females ≥40 and ≤80 years of age |
| Criteria | Able to provide written informed consent |
| | Diagnosis of acute exacerbation of moderate to severe COPD with onset of |
| | acute exacerbation within 1 week of enrollment |
| | Pulmonary Function: FVC < 80% and >30% predicted, FEV₁/FVC < 0.7, |
| | DLCO < 80% predicted |
| | Post-albuterol/salbutamol FEV1 of less than or equal to 70% of predicted |
| | Current or ex-smoker, with a cigarette smoking history of ≥10 years or >10 |
| | pack-years |
| | No change in medications for COPD in the 60 days prior to the Screening |
| | Visit. |
| | Exclusion Criteria |
| | Requirement for supplemental oxygen at rest |
| | Inability to perform a six-minute walk test |
| | Diagnosis of asthma or other clinically relevant lung disease other than |
| | COPD (e.g. restrictive lung diseases, sarcoidosis, tuberculosis, idiopathic |
| | pulmonary fibrosis, bronchiectasis, or lung cancer) |
| | Subject has a body mass greater than 150 kg (330 lb) or less than 40 kg (88 |
| | lb) |
| | Subject has active infection not responding to therapy |
| | Subject with clinically relevant uncontrolled medical condition not |
| | associated with COPD |
| | Subject has documented history of uncontrolled heart failure |
| | Subject has pulmonary hypertension due to left heart condition |
| | Subject has atrial fibrillation or significant congenital heart defect/disease |
| | Subject has initiated pulmonary rehabilitation within 3 months of screening |
| | Cancer requiring chemotherapy or resection within the 5 years prior to the |
| | Screening Visit (other than basal cell carcinoma) |
| | Biologic medications in the 90 days prior to the Screening Visit |
| | Known history of HIV, or has active Hepatitis B or active Hepatitis C |
| | Pregnant or lactating status. Pregnancy as determined by a positive |
| | pregnancy test prior to procedure |
| | Clinically significant abnormal findings on laboratory screening panels, |
| | including hemoglobin ≤10 g/dL or WBC ≤3 × 10⁹ cells/L |
| | Hepatic dysfunction, as defined as aspartate aminotransferase (AST), |
| | alanine aminotranferase (ALT), or bilirubin levels > 1.5 times the upper |
| | limit of normal range (×ULN) prior to randomization |
| | Chronic renal insufficiency as defined as a serum creatinine > 2.0 mg/dL or |
| | requires dialysis |
| | Organ or stem cell transplantation |
| | Any clinically significant finding that would place the patient at health risk, |
| | impact the study, or affect the completion of the study |
| | Any psychiatric illness that would prevent comprehension of the details and |
| | nature of the study and interfere with follow-up clinic visits |
| | Participation in any experimental drug or device study within the 90 days |
| | prior to the Screening Visit |

### Example 8

### Clinical Trial in Acute Phase of COVID19 Disease

A synopsis of key clinical trial protocol parameters is listed in Table 3, below:

TABLE 3

| | |
|---|---|
| Title | Phase I Multicenter Study to Evaluate the Safety and Efficacy of Intravenous |
| | Infusion of E4ORF1-transduced human umbilical vein endothelial cells for |
| | the Treatment of Subjects With Acute Respiratory Distress Associated with |
| | COVID19 |
| Test Product | E4ORF1-transduced allogeneic human umbilical vein endothelial cells |
| Objective | Safety and feasibility of administration of E-CELs in subjects hospitalized for |
| | COVID-19 until discharge or up to 42 days. |
| | Assess the improvement in respiratory status during hospitalization or up to |
| | 42 days after treatment initiation. |
| Secondary | Assess duration of mechanical ventilation. |
| Objective | PaO2:FiO2 over time. |
| | FiO2 requirement over time |
| | Assess pulmonary function post-extubation until discharge. |
| | Assess duration of oxygen supplementation. |
| | Assess overall mortality until discharge or up to 42 days. |
| | Assess progression to non-respiratory organ failure until discharge or up to |
| | 42 days. |
| | Assess change in Sequential Organ Failure Assessment (SOFA) score. |
| | Assess the incidence of cardiac complications until discharge or up to 42 |
| | days. |
| | Assess the rate of renal complication by increase in serum creatinine until |
| | discharge or up to 42 days. |
| | Assess improvement in acute kidney injury until discharge or up to 42 days. |
| | Measure the reduction in time to clinical improvement, defined as the time |
| | from enrollment to an improvement of two points on an 8-category COVID |
| | ordinal scale or live discharge from the hospital, whichever came first. |
| Design | Subjects who are admitted to the hospital with documented COVID-19 |
| | infection, and require prolonged mechanical ventilation (>5 days) or require |
| | ongoing respiratory support after extubation (>1 day of high-flow or |
| | nonrebreather) and meet eligibility criteria will receive E-CELs. If there are |
| | no Grade 3-4 acute infusion-related reaction after D 0, then subjects will |
| | receive repeat treatment on days 3, 6, and 9. |
| | First 2 subjects will be enrolled serially with a mandatory observation of 12 |
| | days prior to dosing of the next subject. |
| Sample Size | Up to 20 |
| Indication | ARDS associated with COVID19 |
| Visit | Screening (Day −5 to Day −1), Procedure Day (Day 1), and follow up visits at |
| Schedule | weeks 1, 4, 12, 24, 36, and 48 weeks |
| Treatment | E-CELs will be infused intravenously at 10 × 10⁶ cells per kilogram subject |
| | weight on study Day 0, 3, 6, and 9. Shorter duration of therapy is permitted if |
| | subject has resolution of symptoms and is discharged from the hospital. |
| | Subjects will be followed for up to 3 months. |
| Safety | Adverse events, Serious Adverse Events, |
| Inclusion & | Inclusion Criteria |
| Exclusion | Adults ≥18 years old |
| Criteria | Positive for SARS-CoV-2 by PCR |
| | Admitted to the hospital for COVID-19 and has required mechanical |
| | ventilation for at least 5 days and has continuous requirement for mechanical |
| | ventilation or has required mechanical ventilation for any duration, was |
| | extubated, and requires continuous supplemental oxygen for more than one |
| | day following extubation |
| | For female subjects of childbearing potential: A negative serum or urine |
| | pregnancy test at screening is required prior to enrollment.; Subject must be |
| | willing to use a recommended method of contraception from the start of the |
| | screening period and throughout the study period. Note: A female subject is |
| | considered of childbearing potential unless she has had a hysterectomy, |
| | bilateral tubal ligation, or bilateral oophorectomy; has medically documented |
| | ovarian failure (with serum estradiol and follicle-stimulating hormone (FSH) |
| | levels within the institutional laboratory post-menopausal range, and a |
| | negative serum or urine human chorionic gonadotropin (b-HCG); or is |
| | menopausal (age ≥55 years with amenorrhea for ≥6 months). |
| | For males who can father a child and are having intercourse with females of |
| | childbearing potential who are not using adequate contraception: Subject |
| | must be willing to use a recommended method of contraception and refrain |
| | from sperm donation from the start of therapy for at least 1 year after |
| | completion and discussion with treating physician. |
| | Note: A male subject is considered able to father a child unless he has had a |
| | bilateral vasectomy with documented aspermia or a bilateral orchiectomy. |
| | Written or verbal informed consent must be obtained from the subject or their |
| | proxy prior to initiation of any clinical screening procedures that are |
| | performed solely for determining eligibility for research. The procedures and |
| | tests that are to be performed as part of the standard of care which would be |
| | done whether or not study entry was considered may be performed and the |
| | results subsequently used for determining study eligibility without first |
| | obtaining consent. |
| | Any experimental therapy for COVID-19 infection including anti-virals (e.g. |
| | remdesivir), anti-inflammatory (e.g. cytokine or cell signaling inhibitors), and |
| | others are permitted if their use has been approved by the institutional IRB or |
| | has Emergency Use Authorization from FDA |
| | Exclusion Criteria |
| | Subjects with a serious concomitant medical condition that could interfere |
| | with the conduct of the clinical trial, such as: |
| | decompensated liver cirrhosis |
| | unstable angina |
| | congestive heart failure NYHA class III-IV |
| | renal failure requiring dialysis |
| | active confirmed bacterial infection requiring antibiotics |
| | Active Human Immunodeficiency Virus (HIV) infection and Acquired |
| | Immunodeficiency Syndrome (AIDS). |
| | Active malignancy |
| | Solid organ transplant recipients |
| | QTc(F) > 450 ms |
| | Extracorporeal membrane oxygenation |
| | Females who are pregnant or breastfeeding or planning to become pregnant or |
| | breastfeed during treatment and for an additional 30 days or longer after |
| | chemotherapy treatment discontinuation if required by prescribing |
| | information for chemotherapy agents received during the study. |
| | Subjects who have known hypersensitivity reactions to bovine (cow) proteins |
| | or documented allergy to DMSO. |
| | Subject has other conditions that in the opinion of the investigator would |
| | place the subject at increased risk for toxicity by participation in the study. |

### Example 9

### Clinical Trial in Pulmonary Fibrosis

A synopsis of key clinical trial protocol parameters is listed in Table 4, below:

TABLE 4

| | |
|---|---|
| Title | A Phase I Multicenter Study to Evaluate the Safety and Efficacy of E4ORF1- |
| | transduced human umbilical vein endothelial cells for the Treatment of |
| | Subjects With Lung Fibrosis |
| Test Product | E4ORF1-transduced allogeneic human umbilical vein endothelial cells |
| Objective | Safety and feasibility of administration of E-CELs in subjects with lung |
| | fibrosis |
| | Assess the improvement in lung function (e.g.: forced vital capacity) at one |
| | year after treatment |
| Secondary | Assess the response rate (percentage of patients with ≥10% decline in FVC at |
| Objective | one year |
| | Assess performance in six minute walking test at one year |
| | Assess change in lung fibrosis patient reported outcome measure (PRO; e.g .: |
| | “Living with Pulmonary Fibrosis”; “St. George's respiratory questionnaire |
| | SGRQ”) at one year after treatment |
| | Time to first hospitalization for pulmonary disease/respiratory distress |
| Design | Up to 5 sequential dose escalation cohorts |
| | Cohorts will be enrolled sequentially. Within each cohort subjects will be |
| | randomized 4:1 to receive either E-CELs or placebo. E-CEL doses will be as |
| | follows: |
| | Cohort 1: 1 × 10⁶/kg body weight |
| | Cohort 2: 2.5 × 10⁶/kg body weight |
| | Cohort 3: 5 × 10⁶/kg body weight |
| | Cohort 4: 10 × 10⁶/kg body weight |
| | Cohort 5: 20 × 10⁶/kg body weight |
| | Safety data will be assessed by an unblinded DRC after the last subject has |
| | reached W4 before enrollment into subsequent cohorts |
| Sample Size | Up to 20 |
| Indication | Pulmonary fibrosis |
| Visit | Screening (Day −5 to Day −1), Procedure Day (Day 1), and follow up visits at |
| Schedule | weeks 1, 4, 12, 24, 36, and 48 weeks |
| Treatment | E-CELs will be infused intravenously at specified dose on study Day 0 or |
| | Study Day 0 and Day 28 or Study Day 0 and Study Day 28, and Study Day |
| | 56. Shorter duration of therapy is permitted if subject has resolution of |
| | symptoms |
| Safety | Adverse events, Serious Adverse Events, |
| Inclusion & | Inclusion Criteria |
| Exclusion | Adults ≥18 years old |
| Criteria | Diagnosis of pulmonary fibrosis at least 1 year prior to enrollment and no |
| | longer than 5 years prior to enrollment |
| | Confirmation of lung fibrosis by high resolution computed tomography of the |
| | lungs within 6 months of enrollment |
| | Forced vital capacity ≥40% and <80% of predicted normal |
| | FEV₁ ≥ 0.7 |
| | For female subjects of childbearing potential: A negative serum or urine |
| | pregnancy test at screening is required prior to enrollment.; Subject must be |
| | willing to use a recommended method of contraception from the start of the |
| | screening period and throughout the study period. Note: A female subject is |
| | considered of childbearing potential unless she has had a hysterectomy, |
| | bilateral tubal ligation, or bilateral oophorectomy; has medically documented |
| | ovarian failure (with serum estradiol and follicle-stimulating hormone (FSH) |
| | levels within the institutional laboratory post-menopausal range, and a |
| | negative serum or urine human chorionic gonadotropin (b-HCG); or is |
| | menopausal (age ≥55 years with amenorrhea for ≥6 months). |
| | For males who can father a child and are having intercourse with females of |
| | childbearing potential who are not using adequate contraception: Subject |
| | must be willing to use a recommended method of contraception and refrain |
| | from sperm donation from the start of therapy for at least 1 year after |
| | completion and discussion with treating physician. |
| | Note: A male subject is considered able to father a child unless he has had a |
| | bilateral vasectomy with documented aspermia or a bilateral orchiectomy. |
| | Written or verbal informed consent must be obtained from the subject or their |
| | proxy prior to initiation of any clinical screening procedures that are |
| | performed solely for determining eligibility for research. The procedures and |
| | tests that are to be performed as part of the standard of care which would be |
| | done whether or not study entry was considered may be performed and the |
| | results subsequently used for determining study eligibility without first |
| | obtaining consent. |
| | Life expectancy ≥24 months |
| | Exclusion Criteria |
| | Subjects with a serious concomitant medical condition that could interfere |
| | with the conduct of the clinical trial, such as: |
| | Known bacterial infection |
| | decompensated liver cirrhosis |
| | unstable angina |
| | congestive heart failure NYHA class III-IV |
| | renal failure requiring dialysis |
| | active confirmed bacterial infection requiring antibiotics |
| | Active Human Immunodeficiency Virus (HIV) infection and Acquired |
| | Immunodeficiency Syndrome (AIDS). |
| | Active malignancy |
| | Recent history (within 6 months) of severe cardiovascular disease (e.g.: AMI, |
| | severe hypertension, or unstable angina. |
| | Solid organ transplant recipients |
| | Extracorporeal membrane oxygenation |
| | Females who are pregnant or breastfeeding or planning to become pregnant or |
| | breastfeed during treatment and for an additional 30 days or longer after |
| | chemotherapy treatment discontinuation if required by prescribing |
| | information for chemotherapy agents received during the study. |
| | Subjects who have known hypersensitivity reactions to bovine (cow) proteins |
| | or documented allergy to DMSO. |
| | Subject has other conditions that in the opinion of the investigator would |
| | place the subject at increased risk for toxicity by participation in the study. |

We claim:

1. A method of treating lung injury in a mammalian subject in need thereof, the method comprising administering to a subject with lung injury an effective amount of a composition comprising umbilical vein endothelial cells (UVECs), thereby treating the lung injury in the subject

2. The method of claim 1, wherein the umbilical vein endothelial cells (UVECs) are human umbilical vein endothelial cells (HUVECs).

3. The method of claim 2, wherein the HUVECs are E4ORF1+ HUVECs.

4. The method of claim 2, wherein the HUVECs are allogeneic E4ORF1+ HUVECs.

5. The method of any one of claims 1-4, wherein the subject has chronic obstructive pulmonary disease (COPD) and the method results in treatment of the lung injury associated with the COPD in the subject.

6. The method of claim 5, wherein the subject has acute exacerbation of chronic obstructive pulmonary disease (AECOPD) and the method results in treatment of the lung injury associated with the AECOPD in the subject.

7. The method of any one of claims 1-4, wherein the subject has lung injury associated with a viral infection, and the method results in treatment of the lung injury associated with the viral infection in the subject.

8. The method of claim 7, wherein the subject has lung injury associated with a SARS-Cov2 infection, and the method results in treatment of the lung injury associated with the SARS-Cov2 infection in the subject.

9. The method of claim 7, wherein the subject has lung injury associated with influenza virus infection, and the method results in treatment of the influenza virus infection-associated lung injury in the subject.

10. The method of claim 7, wherein the subject has lung injury associated with resipiratory syncytial viral (RSV) infection, and the method results in treatment of the RSV infection-associated lung injury in the subject.

11. The method of any one of the preceding claims, wherein the subject has acute respiratory distress syndrome (ARDS), and the method results in treatment of the ARDS in the subject.

12. The method of any one of claims 8-10, wherein the subject has acute respiratory distress syndrome (ARDS) associated with influenza virus, RSV, or SARS-Cov2 infection, and the method results in treatment of the ARDS in the subject.

13. The method of any one of claims 1-4, wherein the subject has lung injury associated with pulmonary fibrosis, and the method results in treatment of the lung injury associated with the pulmonary fibrosis in the subject.

14. The method of claim 13, wherein the subject has lung injury associated with Idiopathic Pulmonary Fibrosis (IPF), and the method results in treatment of the lung injury associated with the IPF in the subject.

15. The method of any of claims 1-4, wherein the subject has lung injury associated with physical trauma to the lung, and the method results in treatment of the lung injury associated with the physical trauma to the lung.

16. The method of claim 15, wherein the subject has lung injury associated with blunt trauma to the lung, penetrating trauma to the lung, or burns affecting the lung, and the method results in treatment of the lung injury associated with the blunt trauma, penetrating trauma or burns to the lung.

17. The method of claim 15, wherein the subject has lung injury associated with surgical lung resection, and the method results in treatment of the lung injury associated with the lung resection.

18. The method of any one of the preceding claims, wherein the composition comprises UVECs at a concentration of about 5 million cells per ml in a physiological saline.

19. The method of any one of the preceding claims, wherein the composition comprises one or more excipients selected from the group consisting of buffers, salts, polysaccharides, proteins, and preservatives.

20. The method of any one of the preceding claims, wherein the composition comprises human serum albumin (HSA).

21. The method of any one of the preceding claims, wherein the composition comprises Dextran40.

22. The method of any one of the preceding claims, wherein the composition comprises up to about 0.25% DMSO.

23. The method of any one of the preceding claims, wherein the composition is administered to the subject intravenously (IV).

24. The method of any one of the preceding claims, wherein the composition is administered to the subject in a single IV infusion.

25. The method of any one of claims 1-23, wherein the therapeutic composition is administered to the subject in multiple IV infusions.

26. The method of claim 25, wherein the therapeutic composition is administered to the subject in a first IV infusion on Day 0, a second IV infusion on about Day 28, and optionally a third IV infusion on about Day 56.

27. The method of claim 25, wherein the subject has acute respiratory distress syndrome associated with COVID-19 and the therapeutic composition is administered to the subject in a first IV infusion on Day 0, optionally a second IV infusion on about Day 3, optionally a third IV infusion on about Day 6, and optionally a fourth IV infusion on about Day 9.

28. The method of any one of the preceding claims, wherein the composition is administered to the subject at a dose of about 1×10⁶ cells per kg bodyweight, per infusion.

29. The method of any one of claims 1-27, wherein the composition is administered to the subject at a dose of about 5×10⁶ cells per kg bodyweight, per infusion.

30. The method of any one of claims 1-27, wherein the composition is administered to the subject at a dose of about 10×10⁶ cells per kg bodyweight, per infusion.

31. The method of any one of claims 1-27, wherein the composition is administered to the subject at a dose of about 20×10⁶ cells per kg bodyweight, per infusion.

32. The method of any one of the preceding claims, wherein the subject is a human.

33. A composition for use in the method of any one of the preceding claims, wherein the composition comprises UVECs in a physiological saline.

34. The composition of claim 33, wherein the composition comprises UVECs at a concentration of about 5 million cells per ml in a physiological saline.

35. The composition of claim 33 or claim 34, wherein the composition comprises one or more excipients selected from the group consisting of buffers, salts, polysaccharides, proteins and preservatives.

36. The composition of claim 34 or claim 34, wherein the composition comprises human serum albumin (HSA).

37. The composition of claim 33 or claim 34, wherein the composition comprises Dextran40.

38. The composition of claim 33 or claim 34, wherein the composition comprises up to about 0.25% DMSO.

39. The composition of claim 33 or claim 34, wherein the composition comprises human serum albumin (HSA), Dextran40, and up to about 0.25% DMSO.

40. A method for the expansion of pulmonary tissue in vitro, the method comprising culturing the pulmonary tissue in vitro with a population of umbilical vein endothelial cells (UVECs).

41. The method of claim 40, wherein the UVECs are human umbilical vein endothelial cells (HUVECs).

42. The method of claim 41, wherein the HUVECs are E4ORF1+ HUVECs.
